(19) United States
(12) Patent Application Publication
(10) Pub. No.: US 2025/0144275 A1
(43) Pub. Date: May 8, 2025

(54) SYSTEM AND METHOD FOR COATING MEDICAL CONDUIT WITH PARTICLES AND USE THEREOF

(71) Applicant: UNIVERSITY OF PITTSBURGH - OF THE COMMONWEALTH SYSTEM OF HIGHER EDUCATION, Pittsburgh, PA (US)

(72) Inventors: Caroline Nadia FEDOR, Wexford, PA (US); Fuat Baris Bengur, Pittsburgh, PA (US); Kacey Gribbin Marra, Canonsburg, PA (US)

(73) Assignee: UNIVERSITY OF PITTSBURGH - OF THE COMMONWEALTH SYSTEM OF HIGHER EDUCATION, Pittsburgh, PA (US)

(21) Appl. No.: 19/000,330

(22) Filed: Dec. 23, 2024

Related U.S. Application Data
(63) Continuation of application No. PCT/US2023/026464, filed on Jun. 28, 2023.
(60) Provisional application No. 63/356,222, filed on Jun. 28, 2022.

(51) Int. Cl. A61L 31/04; A61L 31/10; A61L 31/16

(52) U.S. Cl. CPC A61L 31/047; A61L 31/10; A61L 31/16; A61L 2300/608; A61L 2420/02

(57) ABSTRACT

The present disclosure relates to a system and method for the production of a medical device for use as a nerve guide. The system and method provide for coating a polymer layer with particles, wherein the particles can comprise active agents for the repair or regeneration of nerve defects. The present invention further provides an implantable device for repairing or regenerating a nerve defect prepared by the system and method set forth above, and methods of treatment using said device.

### CROSS-REFERENCE TO RELATED APPLICATIONS

This application is a continuation of International Application No. PCT/US2023/026464 filed on Jun. 28, 2023, which claims priority to U.S. Provisional Patent Application Ser. No. 63/356,222 filed on Jun. 28, 2022, both of which are hereby incorporated by reference in their entireties.

### GRANT INFORMATION

This invention was made with government support under Grant No. W81XWH-21-1-0480 awarded by the Department of Defense. The government has certain rights in the invention.

### FIELD OF INVENTION

The present disclosure relates to a system and method for coating medical conduits with particles. Said medical conduits can be used to reconstruct nerve defects and/or promote nerve regeneration by delivering one or more active agents such as nerve growth factors.

### BACKGROUND

Extremity and nerve-related injuries can be common injuries sustained in a battlefield. Particularly challenging is the repair of long gap peripheral nerve injuries (e.g., defects >2 cm). Certain treatments (e.g., autograft) can be applied for such injuries. However, existing treatments have certain limitations. For example, autografting requires harvesting and transferring a patient's own nerve. The use of the autograft requires a second surgical site resulting in longer operating times. Furthermore, when a sensory nerve is used to replace a motor/sensory nerve, it can cause permanent numbness in a certain area of the body. If autografting cannot be performed due to a lack of donor tissue, an alternative for injury repair can be a decellularized allograft nerve guide. Commercially available allografts, however, cannot offer the same cellular cues relative to an autograft, where the Schwann cell secretome can be essential for nerve regrowth, resulting in suboptimal repair.

Accordingly, there remains a need for systems and techniques for producing biodegradable constructs that can address the above-mentioned limitations.

### SUMMARY

The present disclosure relates to a novel system and method for the production of biodegradable constructs for use as nerve guides. The disclosed embodiments include methods of coating medical conduits with particles for fabricating the nerve guides. The present disclosure further relates to methods of treatment using the aforementioned biodegradable constructs, wherein the constructs serve as nerve guides for promoting nerve regeneration and repair within a subject.

In certain embodiments, the presently disclosed subject matter provides a system for coating a polymer layer with particles comprising a base, wherein the base comprises a flat surface and a gear track; a removable frame coupled to the plate surface of the base, wherein a layer of particles is formed within the frame; and a mandrel base, wherein a polymer coated mandrel is attached to the mandrel base; wherein the mandrel base rotates in the gear track in a direction perpendicular to the flat surface with the polymer coated mandrel rotating on the layer of particles to form a particle coated polymer layer.

In certain embodiments, the further comprises includes a removable mandrel guide coupled to the base, wherein the mandrel guide forms a channel with the flat surface; when the mandrel base rotates in the gear track, the polymer coated mandrel rotates in the channel.

In certain embodiments, the system further comprises a spreader, wherein the spreader spreads the particles on the flat surface within the frame to form the layer of particles. In certain embodiments, the system further comprises a removable parchment holder coupled to the base, wherein the parchment holder holds wax paper or parchment paper on the flat surface, and wherein the layer of particles is formed on the wax paper or parchment paper.

In particular embodiments of the system, the removable frame is coupled to the flat surface via magnet. In particular embodiments of the system, the gear track is a groove.

In particular embodiments of the system, the particles are selected from the group consisting of a microsphere, a nanosphere, and a combination thereof. In certain embodiments, the particles comprise double-walled particles, wherein the double-walled particles comprise the active agent that is released over a pre-determined period of time. In certain embodiments, the double-walled microspheres comprise a core and shell, wherein the core comprises poly(lactic-co-glycolic acid) (PLGA,) and the shell comprises poly(L-lactide) (PLLA). In certain embodiments, the double-walled microspheres further comprise active agent, and wherein the active agent is Glial Cell Line-Derived Neurotrophic Factor (GDNF). In particular embodiments, the drug dose of the neurotrophic factor in the double-walled microspheres is from about 3 ng/mg to about 6 ng/mg.

In certain embodiments of the system, the mandrel is selected from the group consisting of a biodegradable structure, a biologically derived structure, a bioactive structure, and combinations thereof. In certain embodiments, the mandrel comprises a cylindrical structure of purified collagen or a decellularized scaffold.

In certain embodiments of the system, the polymer is polycaprolactone (PCL). In certain embodiments of the system, the system is made from polylactic acid (PLA) or steel. In certain embodiments, the system is operated manually, semi-automatically, or automatically.

The disclosed subject matter also provides for methods for coating a polymer layer with particles, the method comprising forming a polymer solution; dipping a mandrel in the polymer solution to form a polymer coated mandrel; forming a layer of particles on a flat surface; and rotating the polymer coated mandrel on the layer of particles to form a particle coated polymer layer. In certain embodiments, the polymer coated mandrel is a semi-dry polymer coated mandrel. In certain embodiments, the polymer solution is a biodegradable polymer solution. In certain embodiments, the biodegradable polymer solution comprises polycaprolactone (PCL).

The present invention further provides for, but is not limited to, an implantable device for repairing or regenerating a nerve defect prepared by the system and method set forth above. In certain embodiments, the implantable device is a cylindrical medical device.

The disclosed subject matter further provides for a cylindrical medical device comprising a plurality layer of polymer, wherein at least one of the plurality of polymer layers comprises a coating of a plurality of particles. In certain embodiments, the cylindrical medical device is a nerve guide for regeneration of a nerve defect. In certain embodiments, a thickness of the cylindrical medical device is from about 660 μm to about 790 μm. In certain embodiments, the cylindrical medical device comprises five layers of polymer.

The disclosed subject matter further provides a method for repairing or regenerating a nerve defect in a subject. In certain embodiments, the method comprises placing a cylindrical medical device around the nerve defect.

In certain embodiments of the method, the cylindrical medical device comprises a plurality layer of polymer, wherein at least one of the plurality of polymer layers comprises a coating of a plurality of particles. In certain embodiments, the cylindrical medical device comprises five layers of polymer. In certain embodiments, the polymer is polycaprolactone (PCL).

In certain embodiments of the method, the particles are selected from the group consisting of a microsphere, a nanosphere, and a combination thereof. In certain embodiments, the particles comprise double-walled particles. In certain embodiments, the double-walled particles comprise a core and shell, wherein the core comprises poly(lactic-co-glycolic acid) (PLGA) and the shell comprises poly(L-lactide) (PLLA). In certain embodiments, the double-walled particles comprise an active agent. In certain embodiments, the active agent is Glial Cell Line-Derived Neurotrophic Factor (GDNF). In certain embodiments, a drug dose of the GDNF in the double-walled particles is from about 3 ng/mg to about 6 ng/mg. In certain embodiments, the double-walled particles comprise the active agent that is released over a pre-determined period of time.

### BRIEF DESCRIPTION OF THE DRAWINGS

For a more complete understanding of the present disclosure and its features and advantages, reference is now made to the following description, taken in conjunction with the accompanying drawings.

FIG. 1A is an exemplary photograph of the disclosed system for coating a medical conduit with particles in accordance with certain non-limiting embodiments of the disclosed subject matter. FIG. 1B is a photograph of exemplary embodiments of spreaders in accordance with certain non-limiting embodiments of the disclosed subject matter.

FIGS. 2A-L provide exemplary photographs of coating the medical conduit with particles using the system.

FIG. 3 provides an exemplary method for making a construct in accordance with certain non-limiting embodiments of the disclosed subject matter.

FIG. 4A is a schematic illustration of nerve guide with growth factors for nerve regeneration in accordance with certain non-limiting embodiments of the present disclosed subject matter. FIG. 4B is a schematic cross-section view of a five-layered nerve guide with particles between the first and second layers.

FIG. 5A is an exemplary photograph of a cross-section view of the construct in accordance with certain non-limiting embodiments of the disclosed subject matter. FIG. 5B is an exemplary photograph of a side view of the construct in accordance with certain non-limiting embodiments of the disclosed subject matter. FIG. 5C is an exemplary photograph of a caliper measuring the thickness of the construct in accordance with certain non-limiting embodiments of the disclosed subject matter. FIG. 5D is an exemplary chart of average wall thicknesses of eight guides in relation to polycaprolactone (PCL) viscosities.

FIGS. 6A-6D provide exemplary scanning electron microscope (SEM) images of double-walled microspheres.

FIG. 7 is an exemplary chart of average amount of Glial Cell Line-Derived Neurotrophic Factor (GDNF) encapsulated in double-walled microspheres using 4 μg and 10 μg GDNF.

FIGS. 8A-8F depict a rat surgical model for repairing facial nerve injury. FIG. 8A shows the inferior border of the mandible marked for a 1.5 cm curvilinear incision. FIG. 8B shows the subcutaneous tissues following dissection; the buccal and marginal mandibular branches were identified. The buccal branch was cut and primarily repaired (star), while a 5 mm segment was resected from the marginal mandibular branch and both ends were ligated with sutures (arrows). FIG. 8C shows the nerve guide was placed around the repaired buccal branch and secured with a suture (star) to the underlying fascia. FIG. 8D-8F show repaired nerves in various experimental conditions at the 12-week endpoint: 8D demonstrates transection and repair only, 8E demonstrates transection and repair with empty nerve guide, and 8F demonstrates transection and repair with GDNF-containing guide. Scale bars: 1 cm. GDNF, glial cell line-derived neurotrophic factor.

FIGS. 9A-9C depict whisker movement measurements following surgery. FIG. 9A depicts still images showing the sagittal midline, determined by extending a perpendicular line from the middle point of the medial angle of both eyes toward the nose. The rostrally open angle of the marked whiskers with the midline point was determined by selecting the base of the whiskers and the black marking about 1 cm distal on the whiskers. Representative measurements of the protraction and retraction angles on both sides at the baseline can be visualized. Arrows indicate the C1 whiskers on each side that were marked with a black marker, prior to the measurements. The contrast of the black color with the whiskers allowed for accurate quantification of the movements. FIG. 9B shows the percent recovery of the whisking amplitude from the initial week after the surgery to 6- and 12-week timepoints (n=5 for each group). FIG. 9C shows the longitudinal recovery of both groups with two-point moving average. Error bars represent standard deviation. *p<0.05, **p<0.01, ***p<0.001. ^{Δ}One animal was not able to complete the whisking measurements. n.s., not significant.

FIGS. 10A-10C show compound muscle action potential (CMAP) measurements following surgery. (n=5 for each group). FIG. 10A depicts a representative endpoint still image of the buccal branch exposed and the custom-built electrode cuffs placed distal to the nerve guide (star). Subdermal needle electrodes were placed into the vibrissal muscles on rows C and D of whiskers on the mystacial pad. Scale bar: 1 cm. FIG. 10B shows the average representative waveforms of CMAP recorded from the whisker pads of rats in all experimental conditions. FIG. 10C shows the mean peak-to-peak amplitude of CMAP in all experimental conditions (n=5 for each group). Error bars represent standard error. ***p<0.001.

FIGS. 11A and 11B depict muscle fiber count analysis. FIG. 11A depicts representative images of the levator labii superioris muscles in the denervated sides in all three experimental conditions; stained with Gardner's Trichrome (top) and H&E (bottom). Scale bar: 100 μm. FIG. 11B depicts the mean surface area of the muscle fibers (n=5 for each group). Error bars represent standard error. *p<0.05, ***p<0.001.

FIGS. 12A-12D depict the immunofluorescence analysis of nerve samples. FIG. 12A shows representative immunofluorescence images of nerve samples stained with NEFH (green) for axons, S100 (red) for Schwann cells, and DAPI (blue) for nuclei. Scale bars: 100 μm. FIG. 12B depicts a graph showing the quantification of the axonal count on the region distal to the injury (n=3 for each group). FIG. 12C depicts a graph showing the quantification of the Schwann cell count on the region distal to the injury. FIG. 12D depicts a graph showing the quantification of the ratio of Schwann cells at the area distal to the injury. Error bars represent standard error. *p<0.05, **p<0.01, ***p<0.001.

### DETAILED DESCRIPTION

The present disclosure relates to a system and methods for coating medical conduits with particles for fabricating nerve guide. For purposes of clarity of disclosure and not by way of limitation, the detailed description is divided into the following subsections:
1. Definitions;
2. System for coating particles;
3. Method of making device for regeneration of nerve defects; and
4. Device for regeneration of nerve defects.

### 1. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the disclosure and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open-ended terms.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “allograft” refers to a tissue graft from a donor of the same species as the recipient but genetically identical. For example, but not by limitation, the allograft tissue can include bone, bone marrow, kidney, liver, lung, corneal, pancreas, intestine, blood, uterus, thymus, ovary, tendons, ligaments, skin and heart valves.

The term “biomaterial” refers to a material that has properties that are adequate for mammalian body reconstruction, medical device construction, and/or drug control/release devices or products. This term includes absorbable devices and products, absorbable fabrics or meshes, absorbable adhesives, and absorbable drug control/release devices) as well as non-absorbable devices and products, (e.g., implantable repair, contact lens, or support meshes). The term “absorbable” as used herein refers to materials that will be degraded and subsequently absorbed by the body. The term “non-absorbable” as used herein refers to materials that will not be degraded and subsequently absorbed by the body.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of,” and “consisting essentially of,” the embodiments or elements presented herein, whether explicitly set forth or not.

The term “decellularized organ” as used herein refers to an organ or part of an organ from which the entire cellular and tissue content has been removed, leaving behind a complex interstitial structure. Organs are composed of various specialized tissues. The specialized tissue structures of an organ are the parenchyma tissue, and they provide the specific function associated with the organ. Most organs also have a framework composed of unspecialized connective tissue that supports the parenchyma tissue. The process of decellularization removes the parenchyma tissue, leaving behind the three-dimensional interstitial structure of connective tissue, primarily composed of collagen. The interstitial structure has the same shape and size as the native organ, providing the supportive framework that allows cells to attach to and grow on it. Decellularized organs can be rigid, or semi-rigid, having an ability to alter their shapes. Examples of decellularized organs include, but are not limited to, the heart, nerve, kidney, liver, pancreas, spleen, bladder, ureter, and urethra.

As used herein, the term “effective amount” refers to that amount of active agent (e.g., Glial Cell Line-Derived Neurotrophic Factor (GDNF)) sufficient to treat, prevent, or manage a disease. Further, a “therapeutically effective amount” can mean the amount of active agent alone, or in combination with other therapies, that provides a therapeutic benefit in the treatment or management of the disease, which can include a decrease in severity of disease symptoms, an increase in frequency and duration of disease symptom-free periods, or a prevention of impairment or disability due to the disease affliction. The term can encompass an amount that improves overall therapy, reduces or avoids unwanted effects, or enhances the therapeutic efficacy of or synergies with another therapeutic agent.

As used herein, a “mandrel” can refer to a shaped object that can be inserted into a certain workpiece. For example, the mandrel can comprise glass, metal, a polymer, a biocompatible component, a biological or biologically derived component, or combinations thereof. The mandrel can be adapted to press-fit into a workpiece or fixed by some other means into the workpiece.

As used herein, a “mechanism” refers to a device that transforms input forces and/or movements into a desired set of output forces and movements. For example, the mechanism can include a motor, a cylinder, a generator, a transformer, a turbine, a piston, or combinations thereof.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. Ranges disclosed herein, for example, “between about X and about Y” are, unless specified otherwise, inclusive of range limits about X and about Y as well as X and Y. With respect to sub-ranges, “nested sub-ranges” that extend from either endpoint of the range are specifically contemplated. For example, a nested sub-range of an exemplary range of 1 to 50 can include 1 to 10, 1 to 20, 1 to 30, and 1 to 40 in one direction, or 50 to 40, 50to 30, 50 to 20, and 50 to 10 in the other direction. Ranges disclosed herein, for example, “between about X and about Y” are, unless specified otherwise, inclusive of range limits about X and about Y as well as X and Y.

A “subject” herein can be a human or a non-human animal, for example, but not by limitation, rodents such as mice, rats, hamsters, and guinea pigs; rabbits; dogs; cats; sheep; pigs; goats; cattle; horses; and non-human primates such as apes and monkeys, etc.

The terms “treat,” “treating” or “treatment,” and other grammatical equivalents as used herein, include alleviating, abating, ameliorating, or preventing a disease, condition or symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder.

As used herein, the term “uniform” or “homogeneous” refers to a dispersion of particles on a polymer layer being even. A “uniform” or “homogeneous” dispersion cannot depend upon the density of the particles on the polymer layer.

### 2. System for Coating Particles

The disclosed subject matter provides a system for coating particles, wherein the particles can comprise active agents for regeneration of nerve defects. In certain embodiments, the disclosed system comprises a microcontroller, wherein the system can be operated manually, semi-automatically, or automatically controlled by the microcontroller. In non-limiting embodiments, the microcontroller can monitor, record, and save data related to conditions of the disclosed system.

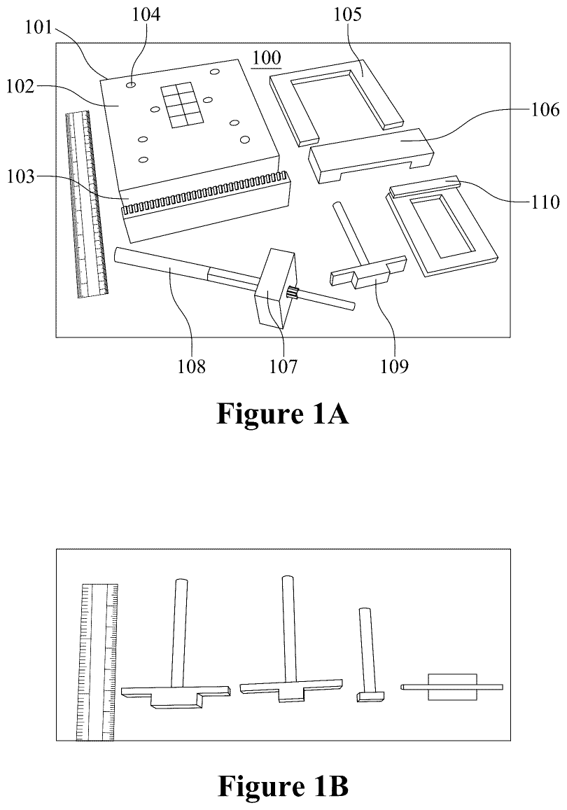

In certain embodiments, as shown in FIG. 1A, the disclosed subject matter provides a system 100 for coating a polymer layer with particles, wherein the system 100 comprises at least a base 101; a removable frame 110; and a mandrel base 107. In certain non-limiting embodiments, the base 101 comprises a flat surface 102 and a gear track 103. In certain embodiments, the gear track is a groove 103. In one embodiment, a mandrel 108 is coupled to the mandrel base 107. For example, the mandrel 108 can be mounted onto the mandrel base 107. In certain embodiments, the mandrel base 107 is configured to rotate in the groove 103 in a direction perpendicular to the flat surface 102.

In some embodiments, the system 100 further comprises a removable mandrel guide 106. In some embodiments, the system 100 further comprises a removable parchment holder 105. In certain embodiments, the system 100 further comprises one or more spreader or stamper 109. More exemplary embodiments of the spreaders are illustrated in FIG. 1B.

In non-limiting embodiments, the system 100 can comprise any suitable material, such as glass, metal, plastic, or combinations thereof. For example, the system 100 can include polylactic acid (PLA). As another example, the system 100 can include steel. In some embodiments, the system 100 can be produced using a three-dimensional (3D) printer. For example, the at least one component of the system 100 (e.g., the base, the removable frame, the mandrel base, spreader or stamper, mandrel guide, parchment holder, etc.) can be designed using the Solid Works CAD/CAM software and printed using a fast filament fusion (FFF) printer.

FIG. 1B illustrates exemplary embodiments of spreaders or stampers. In certain embodiments, the spreaders or stampers can be used to spread particles on the flat surface 102 to form a uniform layer of particles. As discussed above, the spreader or stamper can be produced using 3D printer and can comprise any suitable material, such as glass, metal, plastic, or combinations thereof.

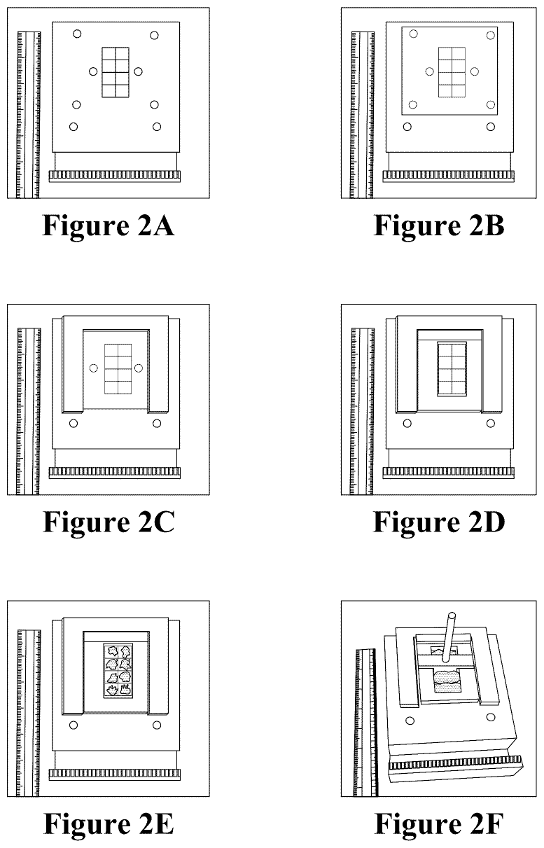

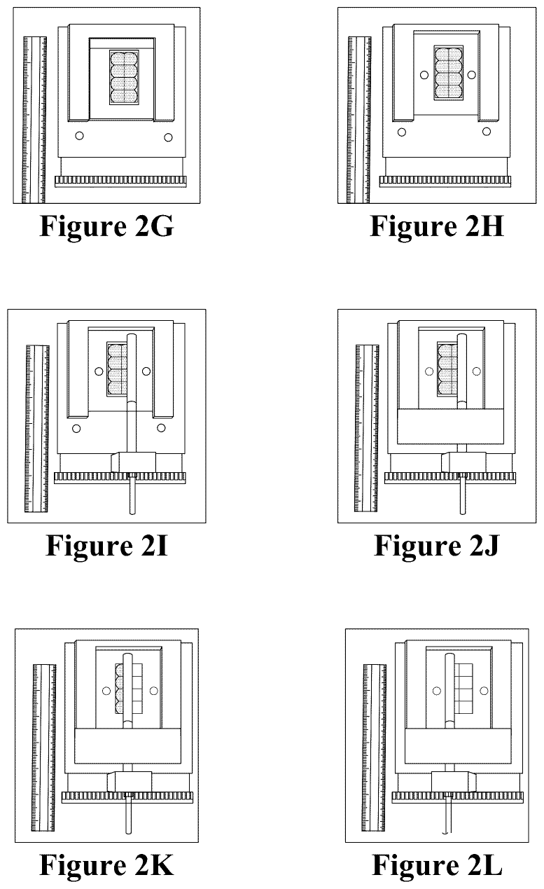

In certain embodiments, the system can comprise a plurality of magnets 104, wherein the plurality of magnets 104 can be used to attach or couple other components (e.g., the removable frame 110, the removable parchment holder 105, the mandrel guide 106, etc.) to the base 101 (FIG. 1A). For example, the parchment holder 105 can be attached or coupled to the base 101 to hold a parchment paper or wax paper on the flat surface (FIGS. 2A-2C). In one example, the parchment paper provides a clean, disposable, and sterile surface for supporting particle layer. In another example, the frame 110 can be attached or coupled to the flat surface and a layer of particles can form within the frame 110 when attached to the flat surface 102 of the base 101 (FIGS. 2D-2H). In another example, the mandrel guide 106 can be coupled to the flat surface and form a channel with the flat surface. In a more specific example, when the mandrel base rotates in the groove, the mandrel rotates in the channel. In one embodiment, the mandrel is coated with a polymer layer prior to rotating on the flat surface. As such, when the polymer coated mandrel rotates on the flat surface with a layer of particles, a particle coated polymer layer is formed on the mandrel (FIGS. 2I-2L).

In certain embodiments, the mandrel can include glass, metal, a biodegradable structure, a biologically derived structure, a bioactive structure, and combinations thereof. For example, the mandrel can include or comprise a cylindrical structure of purified collagen or a decellularized scaffold.

In certain embodiments, as discussed above, the mandrel can be coated with a polymer layer prior to rotating on the flat surface for coating particles. In one embodiment, the polymer coating is formed by dipping the mandrel in a polymer solution (e.g., polycaprolactone (PCL)). In certain embodiments, the nerve guide can be dipped in a PLC solution one or more times. For example, the nerve guide can be dipped at least five dips in a PLC solution. Alternatively, the nerve guide can be dipped at least 10 times, or at least 20 times, or at least 50 times, or at least 75 times. The number of dippings can range from one time to one hundred times. In other embodiments, there is no upper limit of the number of times that the nerve guide can be dipped in the polymer solution. Each dip can be followed with from about 2 minutes to about 20 minutes of drying time. In certain embodiments, the polymer can be a biodegradable polymer. A biodegradable polymer can break down under the conditions of implantation, i.e., in the nervous system tissue environment. The biodegradable polymer and its degradation products can be biocompatible and non-toxic. For example, and not limitation, suitable biodegradable polymers include polycaprolactone (PCL), poly(ester urethane) urea (PEUU), polycarbonate urethane urea (PCUU), poly(ether ester urethane) urea, and other degradable polyurethanes, as well as polylactic acid, poly(lactic-co-glycolic) acid, poly(caprolactone), poly(lactide), acrylic resins, polyglycolide, polylactide, polyhydroxybutyrate, poly (2-hydroxyethyl-methacrylate), poly(ethylene glycol), polydioxanone, chitosan, hyaluronic acid, hydrogels, and combinations thereof. In other non-limiting embodiments, the device can be based on a non-degradable polymer. For example, and not limitation, such non-degradable polymers include silicone rubber, polyethylene, polypropylene, poly(methyl methacrylate) (PMMA), poly(tetrafluoroethylene) (PTFE), polystyrene, polyethylcyanoacrylate, poly(vinyl chloride) (PVC), polyether ether ketone (PEEK), polyether sulfone (PES), and combinations thereof. In certain embodiments, the polymeric matrix can comprise a single type of polymer or a combination of different polymers, e.g., as a polymer blend and/or copolymer. In certain embodiments, the polymeric matrix can comprise a combination of one or more biodegradable polymer and one or more non-degradable polymer. In certain embodiments, the combination of a biodegradable polymer and a non-degradable polymer can itself be biodegradable. In particular embodiments, the polymeric matrix can contain polylactic acid, poly(lactic-co-glycolic) acid and/or poly(caprolactone).

In certain embodiments, the disclosed particles can include a microsphere, nanospheres, or a combination thereof. In non-limiting embodiments, the disclosed particles can comprise a double-walled particles. For example, the double-walled particles can include an active agent, wherein a poly(lactic-co-glycolic acid) layer forms a core, and a poly(lactide) layer forms a shell of the double-walled microsphere. In a particular example, and not by way of limitation, the core comprises poly(lactic-co-glycolic acid) (PLGA) and the shell comprises poly(L-lactide) (PLLA). The double-walled particles can provide sustained release of the active agent over at least seven days. The selection of the active agent can be made based on the function of the conduit, and the physiological needs of the subject to be treated. For example, and not by way of limitation, active agents that can be incorporated into double-walled microspheres include chemotherapeutic drugs or agents (e.g., doxorubicin and/or cisplatin), immunosuppressive drugs or agents (e.g., tacrolimus), anti-inflammatory drugs or agents (e.g., nonsteroidal anti-inflammatory drugs), insulin, dexamethasone, growth factors (e.g., bone morphogenic protein-2, the transforming growth factor β superfamily of proteins, and/or fibroblast growth factors 1 and 2), antihyperglycemic drugs (e.g., pioglitazone), kinase inhibitors, proteins specific to neural regeneration (e.g., glial cell line-derived neurotrophic factor (GDNF), nerve growth factor (NGF), and/or brain-derived growth factor (BDGF)), or combinations thereof. In certain embodiments, a drug dose of the neurotrophic factor in the double-walled microspheres is from about 1 ng/mg to about 200 ng/mg. In one example, a drug dose of the neurotrophic factor in the double-walled microspheres is from about 1 ng/mg to about 100 ng/mg. In another example, a drug dose of the neurotrophic factor in the double-walled microspheres is from about 3 ng/mg to about 6 ng/mg.

One embodiment of the presently disclosed subject matter provides an system for coating a polymer layer with particles comprising a base, wherein the base comprises a flat surface and a groove; a removable frame coupled to the plate surface of the base, wherein a layer of particles is formed within the frame; and a mandrel base, wherein a polymer coated mandrel is attached to the mandrel base; wherein the mandrel base rotates in the groove in a direction perpendicular to the flat surface with the polymer coated mandrel rotating on the layer of particles to form a particle coated polymer layer.

### 3. Method of Making Device for Regeneration of Nerve Defects

The presently disclosed subject matter also relates to a method of making device for regeneration of nerve guide (e.g., nerve conduit).

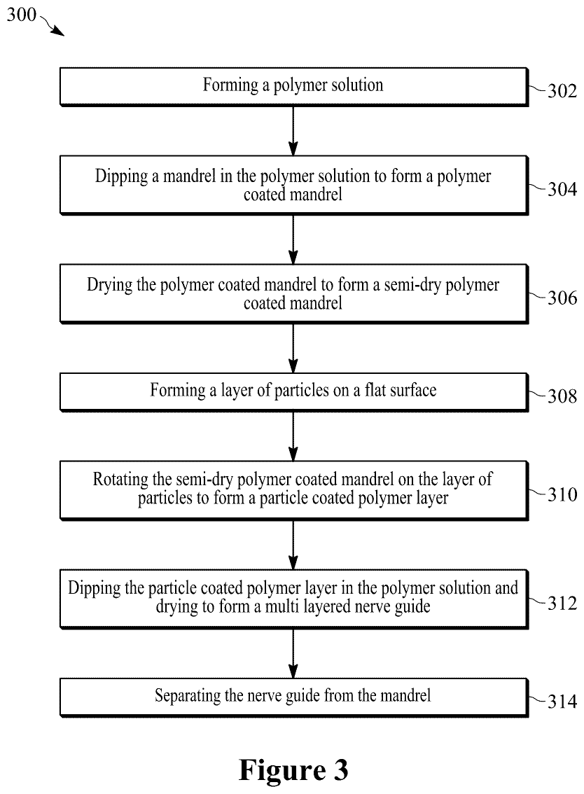

In certain non-limiting embodiments, as shown in FIG. 3, at step 302, the method can comprise forming a polymer solution. In certain embodiments, the polymer can be a biodegradable polymer. A biodegradable polymer can break down under the conditions of implantation, i.e., in the nervous system tissue environment. The biodegradable polymer and its degradation products can be biocompatible and non-toxic. For example, and not limitation, suitable biodegradable polymers include polycaprolactone (PCL), poly(ester urethane) urea (PEUU), polycarbonate urethane urea (PCUU), poly(ether ester urethane) urea, and other degradable polyurethanes, as well as polylactic acid, poly(lactic-co-glycolic) acid, poly(caprolactone), poly(lactide), acrylic resins, polyglycolide, polylactide, polyhydroxybutyrate, poly(2-hydroxyethyl-methacrylate), poly(ethylene glycol), polydioxanone, chitosan, hyaluronic acid, hydrogels, and combinations thereof. In other non-limiting embodiments, the device can be based on a non-degradable polymer. For example, and not limitation, such non-degradable polymers can include silicone rubber, polyethylene, polypropylene, poly(methyl methacrylate) (PMMA), poly(tetrafluoroethylene) (PTFE), polystyrene, polyethylcyanoacrylate, poly(vinyl chloride) (PVC), polyether ether ketone (PEEK), polyether sulfone (PES), and combinations thereof. In certain embodiments, the polymeric matrix can comprise a single type of polymer or a combination of different polymers, e.g., as a polymer blend and/or copolymer. In certain embodiments, the polymeric matrix can comprise a combination of one or more biodegradable polymer and one or more non-degradable polymer. In certain embodiments, the combination of a biodegradable polymer and a non-degradable polymer can itself be biodegradable. In particular embodiments, the polymeric matrix can contain polylactic acid, poly(lactic-co-glycolic) acid and/or poly(caprolactone).

In certain embodiments, the viscosity of the polymer solution can range from about 10 mPa·s to about 300 mPa·s. In one example, the polymer solution is PCL, and the viscosity of the PCL solution is from about 10 mPa·s to about 300 mPa·s. In another example, the viscosity of the PCL solution is from about 50 mPa·s to about 300 mPa·s. In another example, the viscosity of the PCL solution is from about 100 mPa·s to about 200 mPa·s. In another example, the viscosity of the PCL solution is from about 145 mPa·s to about 195 mPa·s.

At step 304, the method 300 can comprise dipping a mandrel in the polymer solution (e.g., PCL) to form a polymer coated mandrel. In one embodiment, the mandrel is a glass mandrel. In one example, the mandrel is a glass mandrel with a polyvinyl alcohol (PVA) coating. In certain embodiments, the mandrel stays in the polymer solution for a period of time before removing the mandrel from the polymer solution. In one embodiment, the period of time can range from seconds to minutes. As an example, the mandrel stays in the polymer solution for about 10 seconds before removing the mandrel from the polymer solution.

At step 306, the method 300 comprises drying the polymer coated mandrel to form a semi-dry polymer coated mandrel. In certain embodiments, the polymer coated mandrel can be dried for an appropriate time in air, for example, for at least about 1 second, at least about a few seconds, at least about 1 minute, at least about 2 minutes, at least about 3 minutes, at least about 4 minutes, at least about 5 minutes or more. In other embodiments, the polymer coated mandrel can be dried in any alternative type of drier appropriate for this purpose for any appropriate period of time to achieve a desired level of dryness.

At step 308, the method 300 comprises forming a layer of particles on a flat surface. In certain embodiments, the layer of particles is formed on the flat surface of the base of the disclosed system shown in FIGS. 2E-2H. In some embodiments, one or more spreader is used to form a uniform layer of particles. See FIGS. 2E-2H. In one embodiment, the particles can include a microsphere, nanospheres, or a combination thereof, as discussed above. In a more specific example, the particles are double-walled microspheres comprising active agent (e.g., GDNF). In some embodiments, the uniform layer of particles can comprise any density depending on the intended use of the conduit. As an example, the density of the layer of particles is from about 1 mg/cm² to about 150 mg/cm². In another example, the density of the layer of particles is from about 10 mg/cm² to about 100 mg/cm². As another example, the density of the layer of particles is from about 20 mg/cm² to about 40 mg/cm². In one example, the density of the layer of particles are determined based on the desired drug dosage of the active agents for a patient.

At step 310, the method 300 comprises rotating the semi-dry polymer coated mandrel on the layer of particles to form a particle coated polymer layer. In certain embodiment, the rotating the semi-dry polymer coated mandrel process is similar to the process discussed in FIGS. 2I-2L discussed above. In certain embodiments, the particle coated polymer layer can comprise a uniform or homogeneous layer of particles. In one example, the uniform layer of particles cannot comprise overlapping of particles on the polymer layer. In another embodiment, the rotating of the mandrel on the layer of particles cannot cause distortion of the semi-dry polymer coating.

At step 312, the method 300 comprises dipping the particle coated mandrel in the polymer solution and then drying for a few times to form a multi layered nerve guide for regeneration of nerve defects. In certain embodiments, before dipping in the polymer solution to form a new polymer layer, the last coated polymer layer on the mandrel is semi-dry. For example, each dip can be followed with 5 minutes of drying time to form semi-dry coating. In other embodiments, before dipping in the polymer solution, the previous formed polymer layer on the mandrel is completely dry. For example, each dip can be followed with at least 10 minutes of drying time to form dry coating. The drying can be done in air or in any appropriate type of drier. Drying times can vary, based on the desired level of dryness. The disclosed method can be repeated until the pre-determined numbers of layers is achieved. In one embodiment, the disclosed method can repeat dipping and removing steps between zero to ninety-nine times. In another embodiment, there is no upper limit of the number of times that the nerve guide can be dipped in the polymer solution. In non-limiting embodiments, the disclosed method can repeat dipping and removing steps between about two to five times. In one example, the disclosed method can repeat dipping and removing steps four more times to obtain a five-layered nerve guide.

In other embodiments, the disclosed methods can be repeated until the pre-determined thickness or number of coating layers is achieved. In non-limiting embodiments, the disclosed system can repeat dipping and removing steps between about six and about ten times to produce a predetermined wall thickness after a lumen is created upon removing the mandrel. The predetermined wall thickness can be about 10 microns, about 50 microns, about 100 microns, about 150 microns, about 200 microns, about 250 microns, about 300 microns, about 350 microns, about 400 microns, about 450 microns, about 500 microns, about 550 microns, about 600 microns, about 650 microns, about 700 microns, about 750 microns, about 800 microns, about 850 microns, about 900 microns, about 950 microns, about 1000 microns, about 2000 microns, or about 3000 microns.

At step 314, the method 300 comprises separating the five-layered nerve guide from the mandrel. In certain embodiments, before coating the mandrel with the polymer solution and the particles, the mandrel is coated with a water-soluble polymer, e.g., polyvinyl alcohol (PVA). After the multi-layered nerve guide is formed, the mandrel can be submerged in water for a period of time, e.g., a few hours, to dissolve the water-soluble polymer to separate the nerve guide from the mandrel.

One embodiment of the presently disclosed subject matter provides a method for coating a polymer layer with particles comprising forming a polymer solution; dipping a mandrel in the polymer solution to form a polymer coated mandrel; forming a layer of particles on a flat surface; and rotating the polymer coated mandrel on the layer of particles to form a particle coated polymer layer.

### 4. Device for Regeneration of Nerve Defects

One embodiment of the presently disclosed subject matter provides an implantable device for regeneration of nerve defects prepared by the system and method set forth above. The device can locally deliver an active agent (e.g., bioactive neurotrophic factor) in physiologically relevant or supraphysiologic concentrations for pre-selected periods (e.g., for at least 50 days). The presently disclosed subject matter is equally applicable to any medical device for which it is desired to deliver an active agent over an extended period of time. The presently disclosed subject matter can be used in a human, non-human primate, non-human mammal, rodent, or other non-human animal subjects.

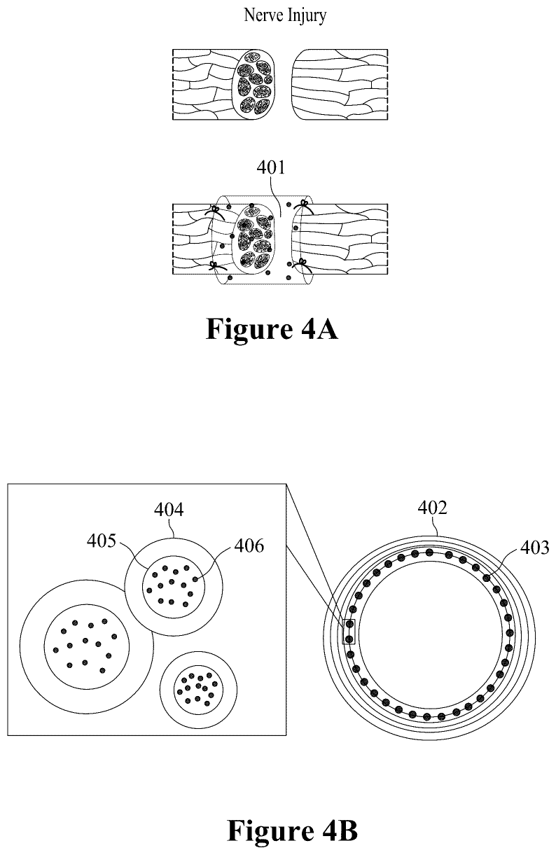

In certain embodiments, as shown in FIG. 4A, the device is a cylindrical medical device. In one embodiment, the cylindrical medical device is a nerve guide or nerve conduit comprising active agent 401 and can locally deliver the active agent 401 in physiologically relevant concentrations for pre-selected periods for regeneration of nerve defect. In non-limiting embodiments, as shown in FIG. 4B, the conduit can comprise multi layers of polymer 402 and a layer of particle 403 coating. In certain embodiments, the conduit comprises five layers of polymer 402 and a layer of particle coating between the first and second layers. As embodied herein, the polymer can be a biodegradable polymer. A biodegradable polymer can break down under the conditions of implantation, i.e., in the nervous system tissue environment. The biodegradable polymer and its degradation products can be biocompatible and non-toxic. For example, and not limitation, suitable biodegradable polymers include polycaprolactone (PCL), poly(ester urethane) urea (PEUU), polycarbonate urethane urea (PCUU), poly(ether ester urethane) urea, and other degradable polyurethanes, as well as polylactic acid, poly(lactic-co-glycolic) acid, poly(caprolactone), poly(lactide), acrylic resins, polyglycolide, polylactide, polyhydroxybutyrate, poly(2-hydroxyethyl-methacrylate), poly(ethylene glycol), polydioxanone, chitosan, hyaluronic acid, hydrogels, and combinations thereof. In other non-limiting embodiments, the device can be based on a non-degradable polymer. For example, and not limitation, such non-degradable polymers include silicone rubber, polyethylene, polypropylene, poly(methyl methacrylate) (PMMA), poly(tetrafluoroethylene) (PTFE), polystyrene, polyethylcyanoacrylate, poly(vinyl chloride) (PVC), polyether ether ketone (PEEK), polyether sulfone (PES), and combinations thereof. In certain embodiments, the polymeric matrix can comprise a single type of polymer or a combination of different polymers, e.g., as a polymer blend and/or copolymer. In certain embodiments, the polymeric matrix can comprise a combination of one or more biodegradable polymer and one or more non-degradable polymer. In certain embodiments, the combination of a biodegradable polymer and a non-degradable polymer can itself be biodegradable. In particular embodiments, the polymeric matrix can contain polylactic acid, poly(lactic-co-glycolic) acid and/or poly(caprolactone).

In certain embodiments, the particles are selected from the group consisting of a microsphere, a nanosphere, and a combination thereof. In one embodiment, the particles 403 are double-walled microspheres. The double-walled microsphere 403 can include a shell 404 and a core 405 comprising a biodegradable polymer. For example, and not limitation, suitable biodegradable polymers include poly(ester urethane) urea (PEUU), polycarbonate urethane urea (PCUU), poly(ether ester urethane) urea, and other degradable polyurethanes, as well as polylactic acid, poly(lactic-co-glycolic) acid, poly(caprolactone), poly(lactide), acrylic resins, polyglycolide, polylactide, polyhydroxybutyrate, poly(2-hydroxyethyl-methacrylate), poly(ethylene glycol), polydioxanone, chitosan, hyaluronic acid, hydrogels, and combinations thereof.

The order of the walls (that is to say, which polymer becomes the core and which polymer becomes the shell) can be determined based on the principles of phase separation. For example, once solutions containing the two polymer “walls” can be mixed to form an emulsion, the polymer layer that is first to precipitate out the solvent associated therewith (i.e., the solvent that is first to evaporate) can form the core layer, and the later-precipitating polymer can form the shell. Persons of ordinary skill in the art can obtain the desired wall order based on, for example, the hydrophilicity of the solvent selected, the polarity of the solvent selected, and the solubility profile of the polymer itself. Phase separation techniques are known to those of ordinary skill in the art, and details can be found, for example, in “In vitro and in vivo degradation of double-walled polymer microspheres,” Journal of Controlled Release 40:169-178 (1996), and “In vitro degradation of polyanhydride/polyester core-shell double-walled microspheres,” International Journal of Pharmaceutics, 301:294-303 (2005), each of which is hereby incorporated by reference in their entirety. Double-walled microspheres 103 can be reproducibly integrated within polymer nerve conduit in manufacturer-controlled distribution. To confirm the distribution of microspheres within the nerve guide, fluorescently labeled bovine serum albumin (BSA) can be encapsulated and visualized through fluorescent microscopy.

In certain non-limiting embodiments, the particles 403 can contain active agents 406. In one embodiment, a double-walled microsphere delivery system is provided for delivery of an active agent 406 (e.g., bioactive GDNF) with a sustained release profile of at least 1, 10, 20, 30, 40, 50, 60, 70, 80, 90, 100, 150, 200, 250, 300, 350, 400, 450, 500, 550, 600, 650, 700, 750, 800, 850, 900, 950, 1000 days or more. In particular embodiments, the double-walled microsphere 403 can release the active agent for at least 80 days. In non-limiting embodiments, the double-walled microsphere 403 can minimize an initial burst release and induce a controlled release of an active agent.

In particular embodiments, double-walled microspheres 403 including poly(L-lactide) (PLLA) and poly(lactic-co-glycolic acid) (PLGA) walls can be incorporated into five layered poly(caprolactone) (PCL) nerve conduits. The PLLA wall can be the shell and the PLGA wall can be core. Alternatively, the PLLA wall can be the core and the PLGA wall can be shell. In certain embodiments, the double-walled microspheres 403 can include GDNF as an active agent.

The device can be suitable for implantation into a subject. The nerve conduit (e.g., nerve guide) can be configured to be applied to an area of nervous tissue for treatment. For example, the device can be applied to a target area in the subject by covering or wrapping the target area with the device by suturing, stapling, adhering with adhesive, tying, or otherwise attaching the device to itself and/or to tissue in the target area. For example, as shown in FIG. 4A, the device can be configured as a sheet that is wrapped around a target nerve and sutured in place.

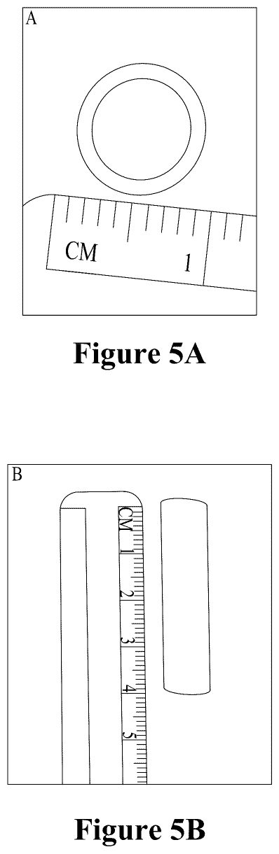

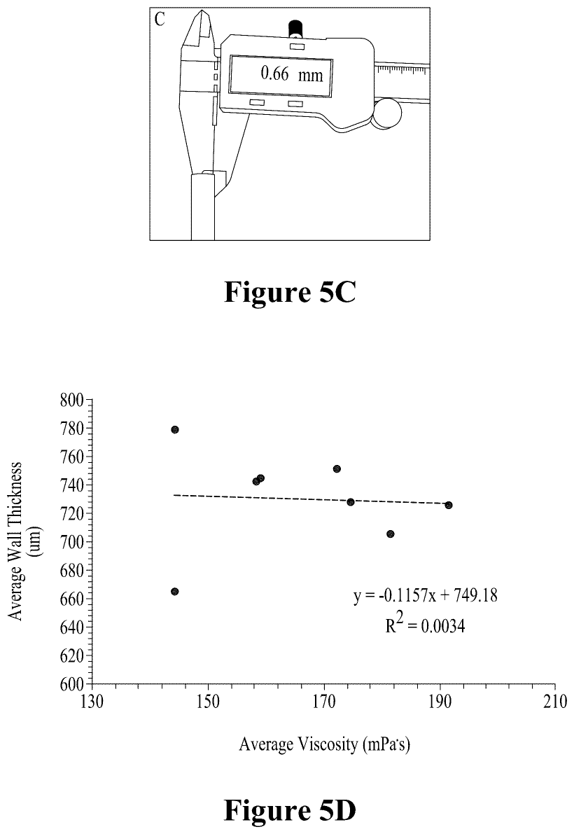

The nerve conduit can have a variety of lengths and diameters depending on source and intended use. In certain embodiments, the nerve conduit can have a length of at least about 5 mm, about 10 mm, or about 50 mm, and up to about 50 mm, about 100 mm, about 500 mm, about 1 cm, about 3 cm, about 5 cm, or about 10 cm or longer. In particular embodiments, the length of the nerve graft can be up to about 4 cm, as shown in FIG. 5B. In certain embodiments, the nerve graft can have a length between about 1 cm and about 8 cm, or between about 2 cm and about 8 cm, or between about 3 cm and about 8 cm.

In certain non-limiting embodiments, the nerve conduit can have a diameter between about 0.5 mm and about 10 mm, or between about 0.5 mm and about 5 mm, or between about 1 mm and about 5 mm. In one example, the nerve conduit can have a diameter ranging from about 500 μm to about 1 cm. In another example, the nerve conduit can have a wall thickness ranging from about 660 μm to about 790 μm, as shown in FIGS. 5A, 5C, and 5D. In certain embodiments, the uniform dispersion of the particle coating can contribute to the uniform wall thickness and outer diameter of the finished nerve conduits.

One embodiment of the presently disclosed subject matter provides cylindrical medical device comprising: a plurality layers of polymer, wherein at least one of the plurality of polymer layers comprise a coating of a plurality of particles.

### 5. Method for Repairing or Regenerating Nerve Defects

Accordingly, in one set of embodiments, the present invention provides for a method repairing or regenerating a nerve defect in a subject in need of such treatment, comprising implanting, to the subject, a medical device for which it is desired to deliver an effective amount of an active agent over an extended period of time. While the presently disclosed subject matter will be, for convenience, largely discussed with reference to the use of a nerve guide, the presently disclosed subject matter is equally applicable to the use of any medical device for which it is desired to deliver any active agent over an extended period of time.

The present invention also relates to methods of treating injuries to nervous system tissue comprising introducing a medical device as described above into an area of injury or disease.

In one embodiment, the never defect suffered by the subject is due to nerve injury. An injury may be caused, for example, by accidental or surgical trauma, infarction, infection, and/or inflammation.

The methods can include treating any type of nervous system tissue where growth of neuronal processes, e.g. axons, may be desirable. In certain non-limiting embodiments, the target nervous system tissue is a nerve which may be a nerve of the CNS such as a cranial nerve or spinal nerve or may be a peripheral nerve of the PNS. Non-limiting examples of nerves include the abdominal aortic plexus, abducens nerve, accessory nerve, accessory obturator nerve, Alderman's nerve, anococcygeal nerve, ansa cervicalis, anterior interosseous nerve, anterior superior alveolar nerve, Auerbach's plexus, auriculotemporal nerve, axillary nerve, brachial plexus, buccal nerve, cardiac plexus, cavernous plexus, celiac ganglion, cervical plexus, chorda tympani, ciliary ganglion, coccygeal nerve, cochlear nerve, common fibular nerve, common palmar digital nerve, cutaneous nerve, deep fibular nerve, deep petrosal nerve, deep temporal nerves, dorsal scapular nerve, esophageal plexus, ethmoidal nerve, external laryngeal nerve, external nasal nerve, facial nerve, femoral nerve, frontal nerve, gastric plexuses, geniculate ganglion, genitofemoral nerve, glossopharyngeal nerve, greater auricular nerve, greater occipital nerve, greater petrosal nerve, hepatic plexus, hypoglossal nerve, iliohypogastric nerve, ilioinguinal nerve, inferior alveolar nerve, inferior anal nerve, inferior cardiac nerve, inferior cervical ganglion, inferior gluteal nerve, inferior hypogastric plexus, inferior mesenteric plexus, inferior palpebral nerve, infraorbital nerve, infraorbital plexus, infratrochlear nerve, intercostal nerves, intercostobrachial nerve, intermediate cutaneous nerve, internal carotid plexus, internal laryngeal nerve, interneuron, jugular ganglion, lacrimal nerve, lateral cord, lateral pectoral nerve, lateral plantar nerve, lateral pterygoid nerve, lesser occipital nerve, lingual nerve, long ciliary nerve, long thoracic nerve, lower subscapular nerve, lumbar nerve, lumbar plexus, lumbar splanchnic nerve, lumboinguinal nerve, lumbosacral plexus, lumbosacral trunk, mandibular nerve, masseteric nerve, maxillary nerve, medial cord, medial cutaneous nerve, medial pectoral nerve, medial plantar nerve, medial pterygoid nerve, median nerve, Meissner's plexus, mental nerve, middle meningeal nerve, motor nerve, musculocutaneous nerve, mylohyoid nerve, nasociliary nerve, nasopalatine nerve, nerve of pterygoid canal, nerve to obturator internus, nerve to quadratus femoris, nerve to the piriformis, nerve to the stapedius, nerve to the subclavius, nervus intermedius, nervus spinosus, nodose ganglion, obturator nerve, occipital nerve, oculomotor nerve, olfactory nerve, ophthalmic nerve, optic nerve, otic ganglion, ovarian plexus, palatine nerve, pancreatic plexus, patellar plexus, pelvic splanchnic nerves, perforating cutaneous nerve, perineal nerve, petrous ganglion, pharyngeal nerve, pharyngeal plexus, phrenic nerve, phrenic plexus, posterior auricular nerve, posterior cord, posterior scrotal nerve, posterior superior alveolar nerve, prostatic plexus (nervous), pterygopalatine ganglion, pudendal nerve, pudendal plexus, radial nerve, recurrent laryngeal nerve, renal plexus, sacral plexus, sacral splanchnic nerves, saphenous nerve, sciatic nerve, semilunar ganglion, sensory nerve, short ciliary nerve, sphenopalatine nerve, splenic plexus, subcostal nerve, submandibular ganglion, suboccipital nerve, superficial fibular nerve, superior cardiac nerve, superior cervical ganglion, superior gluteal nerve, superior hypogastric plexus, superior labial nerve, superior laryngeal nerve, superior mesenteric plexus, superior rectal plexus, supraclavicular nerve, supraorbital nerve, suprarenal plexus, suprascapular nerve, supratrochlear nerve, sural nerve, sympathetic trunk, thoracic aortic plexus, thoracic splanchnic nerve, thoraco-abdominal nerve, thoracodorsal nerve, tibial nerve, transverse cervical nerve, trigeminal nerve, trochlear nerve, tympanic nerve, ulnar nerve, upper subscapular nerve, uterovaginal plexus, vagus nerve, ventral ramus, vesical nervous plexus, vestibular nerve, vestibulocochlear nerve, zygomatic nerve, zygomaticofacial nerve, and zygomaticotemporal nerve. In particular embodiments, the nervous system tissue is the sciatic nerve. In particular embodiments, the nervous system tissue is a bundle of axons in the spinal cord.

In certain embodiments, the invention provides for a method of treating an injury to a nerve, wherein a proximal and a distal end of the nerve are separated by a gap, comprising introducing, into the gap. a nerve guide as described herein. In certain non-limiting embodiments, the nerve guide, when placed, covers at least 50 percent, or at least 75 percent, or at least 80 percent, or at least 90 percent, of the gap between the proximal and distal nerve ends. In certain non-limiting embodiments, the gap is at least about 1 cm. In certain non-limiting embodiments, the gap is at least about 2 cm. In certain non-limiting embodiments, the gap is at least about 3 cm. In certain non-limiting embodiments, the gap is at least about 4 cm. In certain non-limiting embodiments, the gap is at least about 5 cm. In certain non-limiting embodiments, the gap is up to 3 cm. In certain non-limiting embodiments, the gap is up to 4 cm. In certain non-limiting embodiments, the gap is up to 5 cm. In certain non-limiting embodiments, the gap is up to 6 cm. In certain non-limiting embodiments, the gap is up to 8 cm. In certain non-limiting embodiments, the gap is up to 10 cm.

In certain embodiments, the invention provides for a method of treating an injury to a nerve, wherein a proximal and a distal end of the nerve are separated by a gap, comprising introducing, into the gap. a composite nerve guide as described herein. In certain non-limiting embodiments, the composite nerve guide, when placed, covers at least 50 percent, or at least 75 percent, or at least 80 percent, or at least 90 percent, of the gap between the proximal and distal nerve ends. In certain non-limiting embodiments, the gap is at least about 1 cm. In certain non-limiting embodiments, the gap is at least about 2 cm. In certain non-limiting embodiments, the gap is at least about 3 cm. In certain non-limiting embodiments, the gap is at least about 4 cm. In certain non-limiting embodiments, the gap is at least about 5 cm. In certain non-limiting embodiments, the gap is up to 3 cm. In certain non-limiting embodiments, the gap is up to 4 cm. In certain non-limiting embodiments, the gap is up to 5 cm. In certain non-limiting embodiments, the gap is up to 6 cm. In certain non-limiting embodiments, the gap is up to 8 cm. In certain non-limiting embodiments, the gap is up to 10 cm.

In certain embodiments, the invention provides for a method of promoting axonal regrowth, wherein a proximal and a distal end of a group of axons are separated by a gap, comprising introducing, into the gap. a composite nerve guide as described herein. In certain non-limiting embodiments, the composite nerve guide, when placed, covers at least 50 percent, or at least 75 percent, or at least 80 percent, or at least 90 percent, of the gap between the proximal and distal nerve ends. In certain non-limiting embodiments, the gap is at least about 1 cm. In certain non-limiting embodiments, the gap is at least about 2 cm. In certain non-limiting embodiments, the gap is at least about 3 cm. In certain non-limiting embodiments, the gap is at least about 4 cm. In certain non-limiting embodiments, the gap is at least about 5 cm. In certain non-limiting embodiments, the gap is up to 3 cm. In certain non-limiting embodiments, the gap is up to 4 cm. In certain non-limiting embodiments, the gap is up to 5 cm. In certain non-limiting embodiments, the gap is up to 6 cm. In certain non-limiting embodiments, the gap is up to 8 cm. In certain non-limiting embodiments, the gap is up to 10 cm.

An effective dose/amount may be calculated by determining the amount needed to be administered to produce a concentration sufficient to achieve the desired effect in the tissue to be treated, taking into account, for example, route of administration, bioavailability, half-life, and the concentration which achieves the desired effect in vitro or in an animal model system, using techniques known in the art.

The method of the present disclosure may be applied to a human, non-human primate, non-human mammal, rodent, or other non-human animal subject.

In one embodiment, summarized in greater detail in the Examples below, a method for repairing or g regenerating a nerve, comprising implantation of a cylindrical medical device wherein the devices comprises a plurality layer of polymer and a double-walled particle delivery system for delivery of an active agent (e.g., bioactive GDNF) with a sustained release profile of at. In this particular embodiment, particles or microspheres, preferably double-walled particles, are incorporated within a degradable poly(caprolactone) nerve guide in a reproducible distribution. Implantation of nerve guides across a 1.5 cm defect in a rat sciatic nerve gap resulted in an increase in tissue integration in both the proximal and distal segments of the lumen of the nerve guide after 6 weeks. In addition, transverse sections of the distal region of the explanted guides showed the presence of Schwann cells while none were detectable in negative control guides. Migration of Schwann cells to double-walled microspheres indicated that bioactive GDNF was encapsulated and delivered to the internal environment of the nerve guide. Because GDNF increased tissue formation within the nerve guide lumen and also promoted the migration and proliferation of Schwann cells, the presently disclosed nerve guides can promote nerve regeneration beyond that capable with pre-existing nerve guides.

### EXAMPLES

The presently disclosed subject matter will be better understood by reference to the following Example, which is provided as exemplary of the presently disclosed subject matter, and not by way of limitation.

### Example 1. Development of Nerve Guides

Preparation of the GDNF-containing polymer conduit guide was performed as had been previously described³⁷⁻⁴⁰. Briefly, a poly(caprolactone) solution was prepared in ethyl acetate. This solution was used to prepare the main structure of the nerve guide by vertically dipping a glass mandrel rod of 1.5 mm diameter. Slowly degrading double-walled microspheres were created by mixing poly(lactic-co-glycolic acid) (PLGA; 50:50) and PLLA. Recombinant human GDNF protein (R&D Systems) was mixed with the poly(L-lactic acid) (PLLA) solution. Surface texture of the microspheres was morphologically assessed using scanning electron microscopy (Zeiss SIGMA VP). The amount of GDNF encapsulated in the microspheres was assessed using an Enzyme Linked Immunosorbent Assay (ELISA).

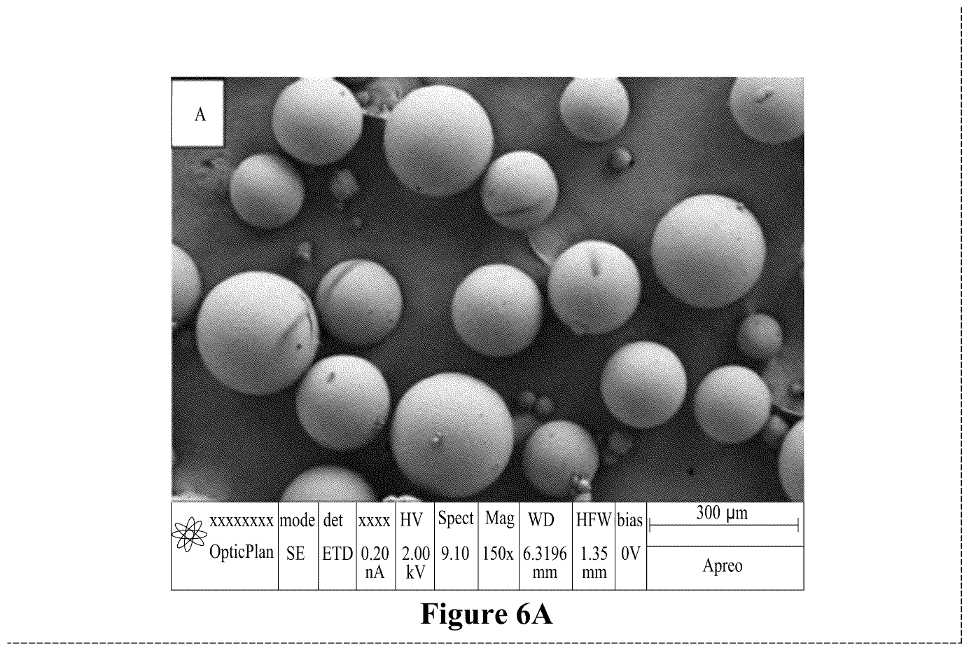

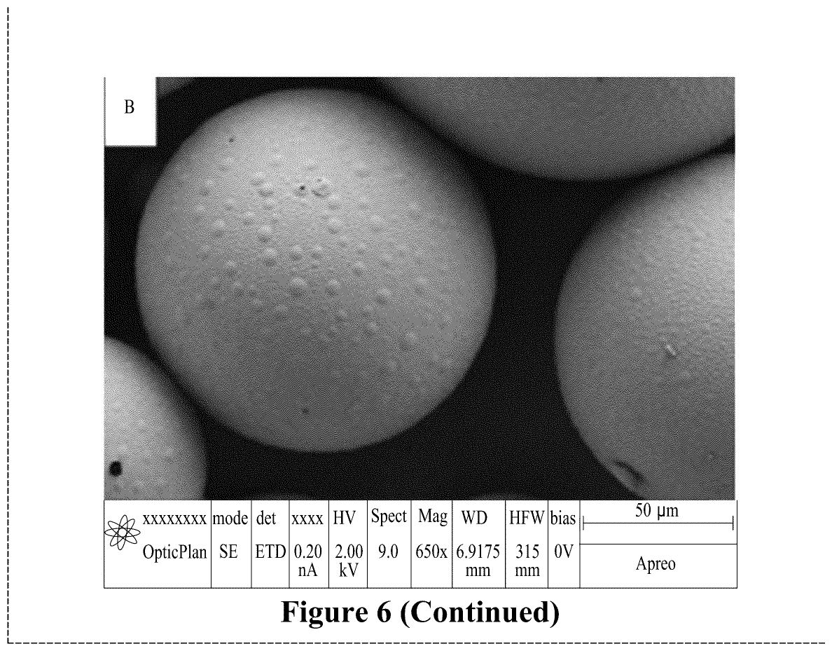

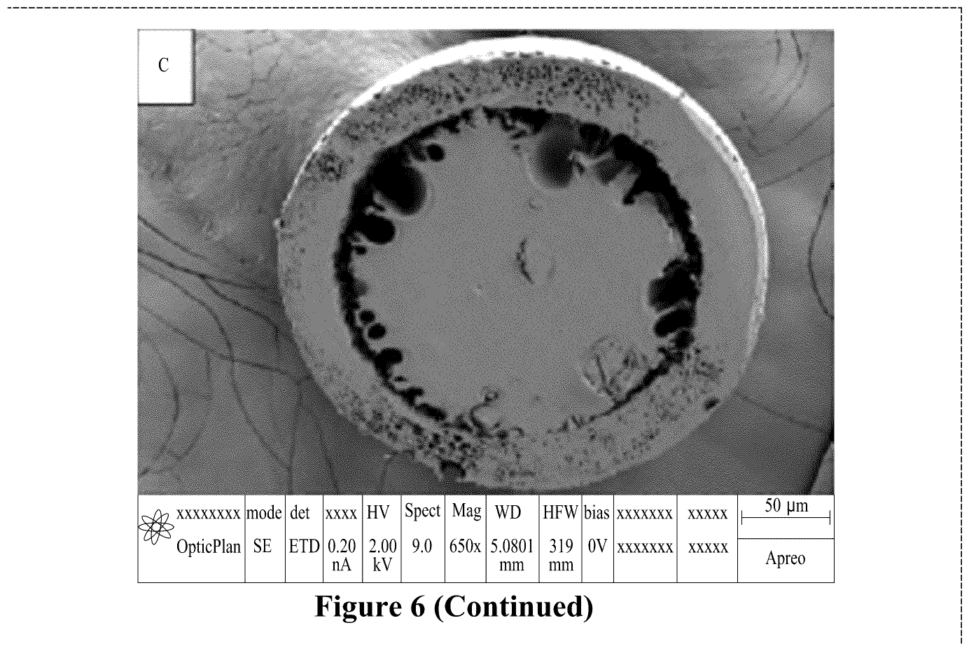

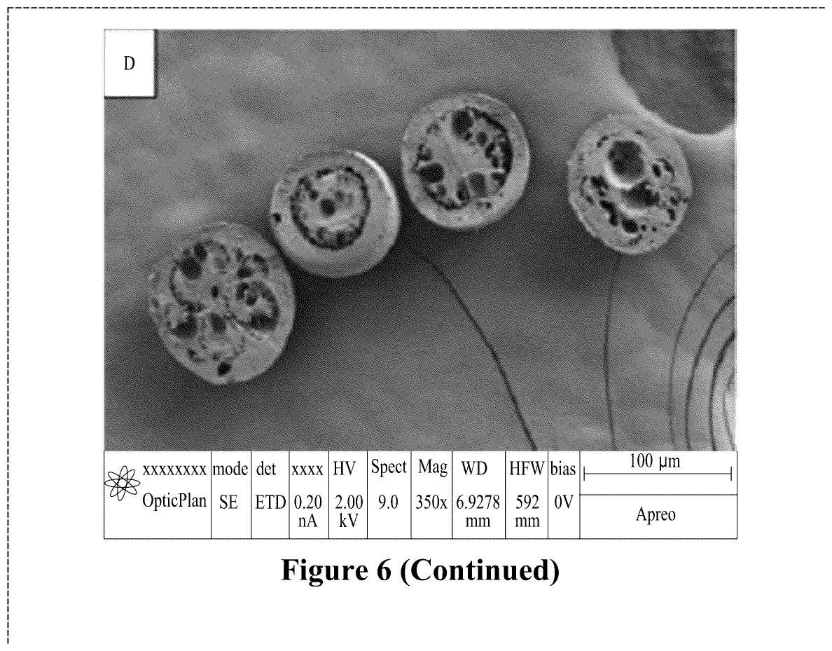

Double-walled microsphere samples were prepared via water-oil-water emulsion technique using various amounts of initial GDNF (4 μg and 10 μg) as active agent. As shown in FIGS. 6A and 6B, the double-walled microspheres have a diameter ranging from about 15 μm to about 280 μm. The double-walled microspheres comprised poly(L-lactide) (PLLA) as the shell and poly(lactic-co-glycolic acid) (PLGA) as the core, as shown in FIGS. 6C and 6D.

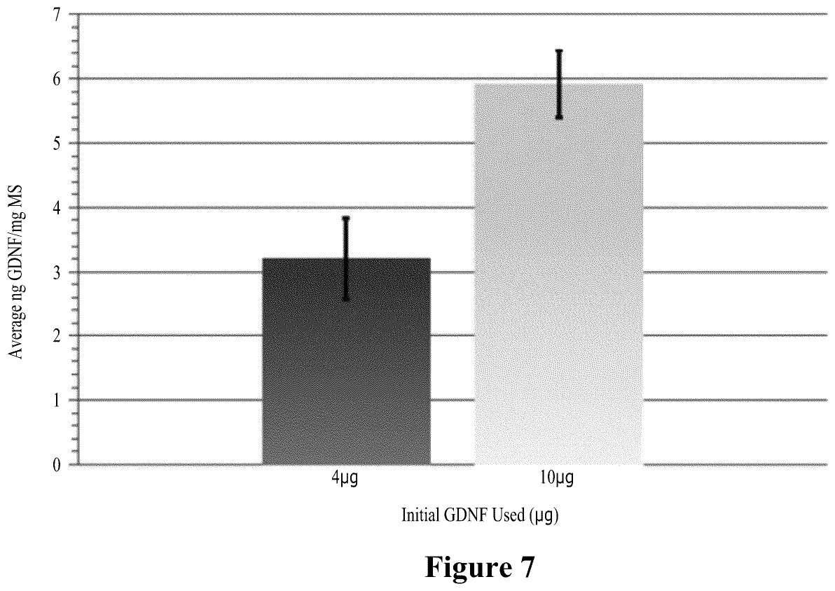

FIG. 7 provides an exemplary chart of the average amount of GDNF encapsulated in double-walled microspheres using 4 μg and 10 μg GDNF. Five samples were tested in each group. As shown, when 4 μg GDNF were used to prepare the doubled-walled microspheres, the drug dose of the double-walled microspheres is about 3 ng/mg. Furthermore, when 10 μg GDNF were used to prepare the doubled-walled microspheres, the drug dose of the double-walled microspheres is about 6 ng/mg.

Next, a layer of the double-walled microspheres were coated on a semi-dry PCL layer similar to the process discussed in FIGS. 2A-2L discussed above. Then, four layers of PCL were coated on the double-walled microsphere coated PCL layer and thus five layered nerve conduit samples were prepared.

As shown in FIGS. 5A, 5C, and 5D, the nerve conduit samples had a wall thickness ranging from about 660 μm to about 790 μm. The uniform dispersion of the particle coating can contribute to the uniform diameter of the finished nerve conduits. Furthermore, the length of the nerve conduit was up to about 4 cm, as shown in FIG. 5B.

### Example 2. Nerve Guide for Repairing Facial Nerve Injury in a Rat Surgical Model

Facial nerve injury and subsequent nerve palsy are associated with functional, psychological, and cosmetic challenges^{1,2}. Nerve palsy can occur due to various etiologies where iatrogenic injuries, trauma, cancer, and benign lesion resections account for more than 30% of all facial nerve injuries³⁻⁷. These injuries can range from transection of the branches of the facial nerve to varying degrees of segmental loss⁸. Surgical intervention is usually preferred in patients with an anatomical disruption of the nerve^{4,8-12} and early repair is critical to prevent muscular atrophy due to denervation^{7,9,13}. Primary tension-free neurorrhaphy of the nerve, where possible, is the gold standard to achieve effective recovery following the injury.

Various nerve conduits are being utilized to facilitate rapid recovery after facial nerve injuries¹⁴⁻¹⁸. The ability to integrate different cues, such as stem cells^{19,20} or neurotrophic factors²¹⁻²³, within the guides is being explored in the preclinical setting²⁴. In addition to physical guidance and support, nerve guides also act as vehicles for local sustained slow release of these cues to accelerate recovery and improve outcomes. Hollow conduits can be utilized as wraps around the repair site to function as protective barriers while minimizing axonal sprouting and scar formation²⁵⁻²⁷.

Glial cell line-derived neurotrophic factor (GDNF) delivery has demonstrated improved recovery in various nerve repair models^{28,29}. GDNF is an endogenous protein secreted by Schwann cells and upregulated in the distal segments of the nerve following denervation to promote Schwann cell proliferation and migration, as well as axonal elongation and branching³⁰⁻³⁴. However, despite being upregulated in the early stages after the injury, expression of the GDNF reduces after its peak at week 1, depriving Schwann cells from their neurotrophic support^{35,36}. The present example demonstrated the use of a nerve guide as a vehicle to achieve sustained release of GDNF in the injury site and compared functional, electrophysiological, and histological outcomes after repair of rat facial nerve transection in control, empty nerve guide, and nerve guide with GDNF conditions.

### Methods

This study was performed on the facial nerve of Lewis rats (Charles River, Wilmington, MA). Institutional approval was obtained before performing the study, and all animal procedures and housing were performed under the guidelines of the University of Pittsburgh Institutional Animal Care and Use Committee and in compliance with animal experimentation ethics policies.

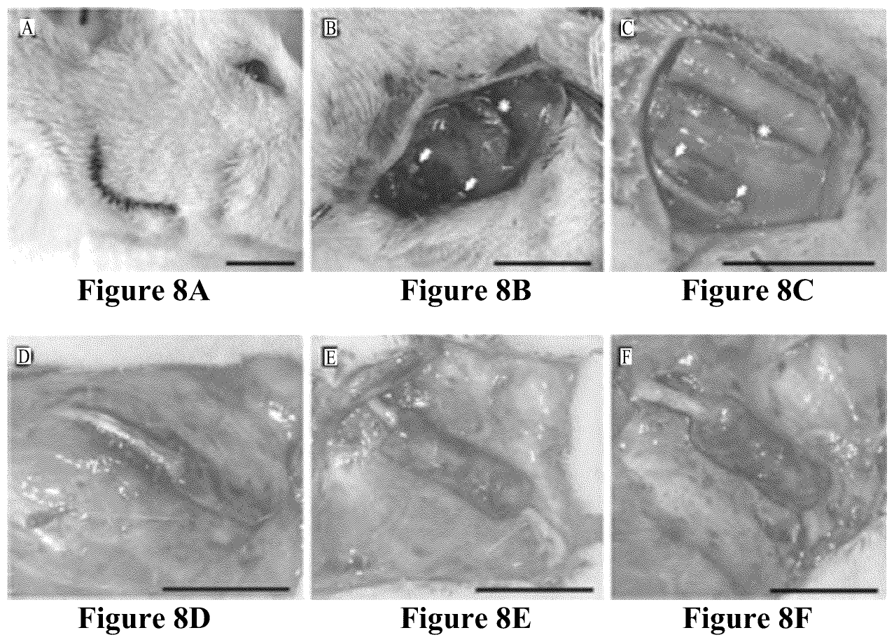

Surgical procedures. Rats were placed under general anesthesia and maintained with Isoflurane. Preauricular/buccal incision lines were marked (FIG. 8A) and the nerve branches were exposed (FIG. 8B). A 5 mm segment of the marginal mandibular branch was removed to prevent cross-innervation contribution to the whisker movement⁴¹. The buccal branch was then transected, and the resultant 2 mm defect was primarily repaired with 9-0 nylon sutures. After the repair, rats were divided into three experimental conditions (n=5/group) as (1) transection and repair only, (2) transection and repair with empty nerve guide, and (3) transection and repair with a GDNF-containing guide. For groups 2 and 3, a 1 cm nerve guide was wrapped around the nerve at the coaptation site and secured to the underlying fascia with a 7-0 nylon suture (FIG. 8C). At the time of sacrifice, the nerve guides were still intact and integrated with the surrounding tissues (FIGS. 8D-8F).

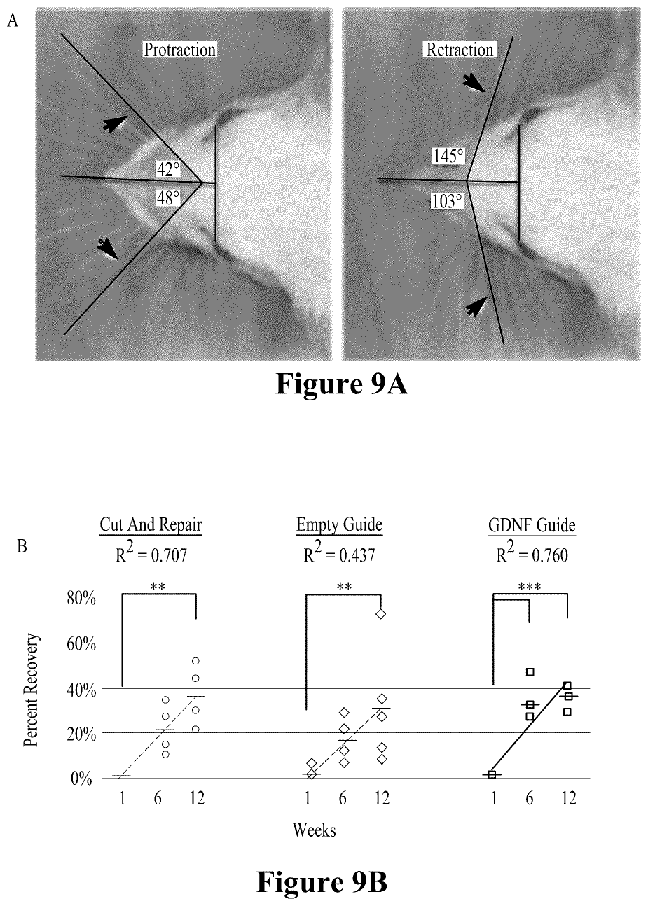

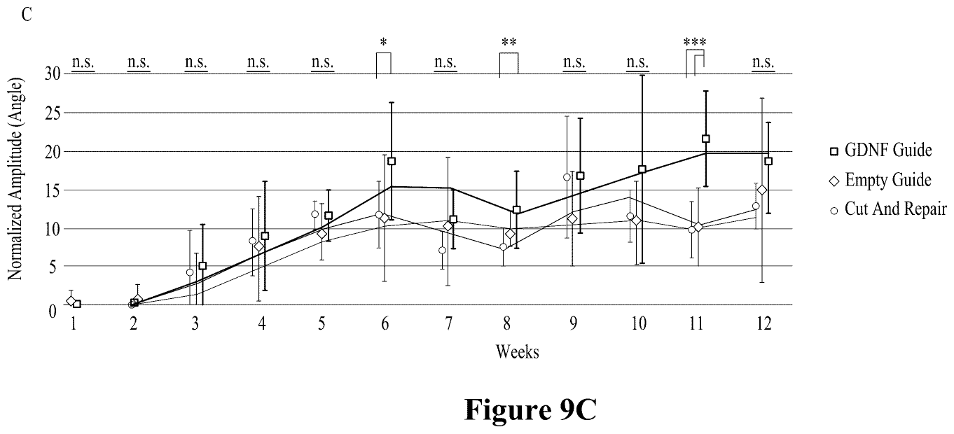

Whisker movements. The difference between maximal protraction and retraction angles was used to determine the amplitude of whisking (FIG. 9A)^{42,43}. Recordings were performed for all rats at baseline and weekly for 12 weeks after the surgery, using a restrainer (Rat Restraint RR-300; IBI Scientific). All measurements were performed twice by two independent blinded observers, and the mean values were analyzed. The amplitude angle of the injured site after the surgery was normalized to the baseline values for each rat.

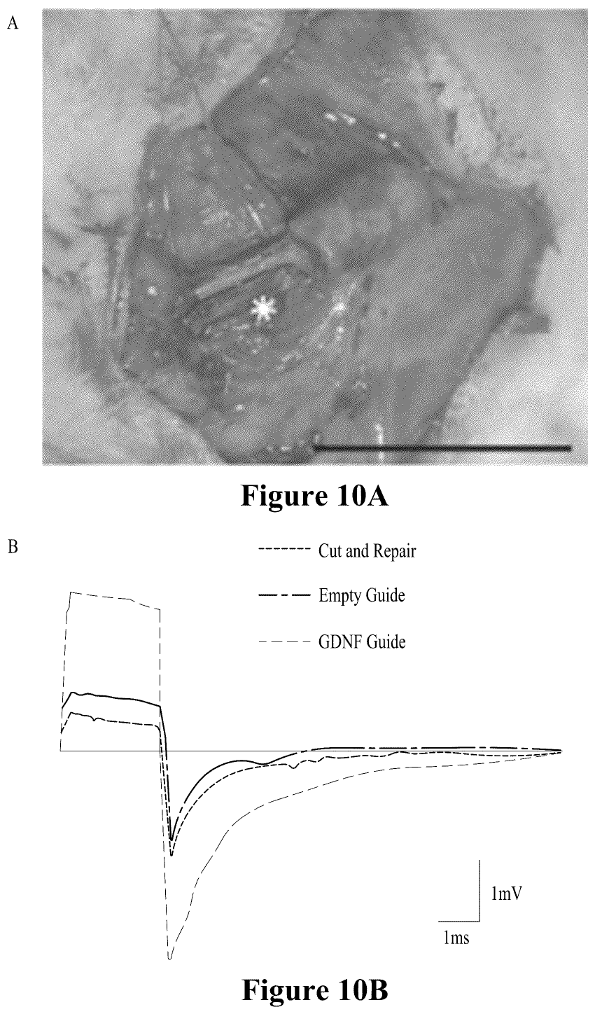

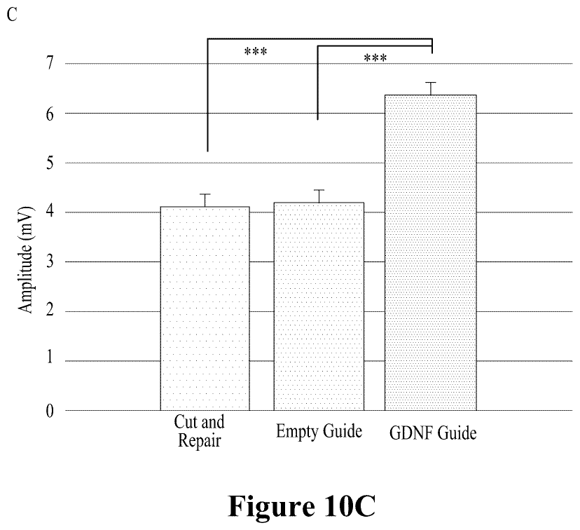

Electrophysiology. At endpoint, rats were deeply sedated with Isoflurane (1.5-2.5%); the sedation was maintained with an intraperitoneal injection of Ketamine, Xylazine, and Acepromazine. Buccal branch was exposed, and custom-built nerve stimulation cuffs were placed on the nerve (FIG. 10A). Two 25G subdermal needle electrodes (RLSND107-1.5, Rhythm Link) were inserted in the targeted vibrissal muscles on rows C and D of whiskers on the mystacial pad.

A grounding probe was placed caudally on the skin. Stimulation at 5 Hz of pulse frequency was induced from the proximal cuff by a stimulus isolator at 0.5 mA over 1000 ms and repeated thrice. Following stimulation, compound muscle action potentials (CMAPs) of the vibrissal muscle were recorded using MyDAQ coded in LabVIEW (National Instruments) at 10,000 Hz sampling rate. Peak-to-peak amplitude for each CMAP waveform (n=15 per animal) was identified as the difference between the highest positive peak and the lowest negative peak.

Systemic GDNF levels. Serum of the rats in the GDNF-treated group (n=3) was collected at the endpoint. The serum samples were analyzed using ELISA as per the manufacturer's instructions to identify systemic expression of GDNF levels (Human GDNF Quantikine ELISA Kit; R&D Systems). The optical density was recorded at 540 nm (Infinite 200 PRO; Tecan).

Histological processing. Samples from the denervated sides were extracted for histological processing. The levator labii superioris muscle was dissected from the frontal bone toward the whisker roots, including all fibers^{44,45}. The mystacial pad was also dissected as a 1×1 cm block to include whisker rows and columns and surrounding intrinsic muscles^{46,47}. The buccal branch of the facial nerve was extracted to include the repaired section and the nerve guide in total. Upon extraction, tissues were cryopreserved and processed for further histomorphometric analysis. For the muscles, the mean muscle fiber pixel area was calculated from brightfield staining. For the nerves, immunofluorescence staining was performed with primary antibodies for S100 (Schwann cells) and neurofilament heavy chain (NEFH, axons). Cross-sections of the main buccal branch distal to the injury site were analyzed for the number of axons. For S100, sections of the nerve distal and proximal to the transection and repair site were quantified separately and the ratio was used for comparison. All samples were blinded before quantification.

Statistical analysis. All data were analyzed using SPSS version 27 (IBM). Normality of the data was verified with Shapiro-Wilk W test before analyses and analysis of variance (ANOVA) with Tukey HSD posthoc was used. Regression analyses were performed to demonstrate trends of recovery and correlations with R²>0.75 considered strong, within 0.75≥R²>0.25 considered moderate, and R²<0.25 considered weak. The animal number was calculated a priori based on previous literature^{38,43,45,48}. A minimum population of n=5 animals was determined to have adequate power to detect an a priori minimum difference of 30% assuming a standard deviation of up to 25% between groups for outcome measures to yield a power of 80%. Significance was assessed based upon rejection of the null hypothesis of equal means at 0.05.

### Results

The present example demonstrated controlled GDNF release from the microspheres. The amount of drug released was 5.5 ng of GDNF per 1 mg of microspheres with an encapsulation efficiency of 22%. Typical nerve guides of 1 cm length and 1.5 mm inner diameter were composed of a total of 41 mg of microspheres to achieve 222 ng of GDNF per guide.

The present example demonstrated GDNF-containing nerve guides restored functional whisker movement after injury. The baseline mean amplitude of whisker movement, i.e., whisking, was 57.09±13.20 before surgery (with mean maximal retraction 106.95±14.11 and mean maximal protraction 49.55±17.05). After the surgery, recovery of the whisker movement became more prominent starting within 3-4 weeks. The GDNF nerve guide animals demonstrated a significantly higher early peak percent recovery at week 6 with 32.47±8.95% (p<0.001), whereas other experimental conditions failed to reach statistical significance in the improvement of whisking amplitude at week 6 (FIG. 9B). All animals demonstrated significant improvement of whisking at 12-week endpoint with the GDNF guide group showing the highest significant increase (p<0.001) relative to the cut and repair (p=0.001) and empty guide (p=0.005) groups.

When the trend of recovery was assessed, R² values trended better toward linear correlation in the GDNF nerve guide group with strong correlation (R²=0.760) versus moderate correlation in empty guide (R²=0.437) and cut and repair only (R²=0.707) groups. The moving average of normalized whisking amplitude demonstrated a similar recovery pattern with cut and repair and empty guide groups, while the GDNF guide group had a higher overall amplitude (FIG. 9C). There was a significant difference at week-11 where the GDNF guide group had the highest normalized whisking amplitude (p<0.001).

The present example demonstrated GDNF-containing guides generated statistically significantly higher mean CMAP compared with all others (p<0.001) (FIGS. 10B and 10C).

The present example demonstrated effective local delivery of GDNF without resulting in measurable systemic levels of GDNF. Rat blood levels were measured at the 12-week timepoint from when the rats received the GDNF-containing nerve guides. ELISA testing demonstrated no detectable levels in the serum.

The present example demonstrated no structural changes to the mystacial pad. The morphologic appearance of the whisker pad was not different across the experimental conditions. Qualitatively, the dermal elements and muscular elements were morphologically similar, demonstrating no local adverse effects of GDNF on the whisker pad and the targeted intrinsic vibrissae muscles.

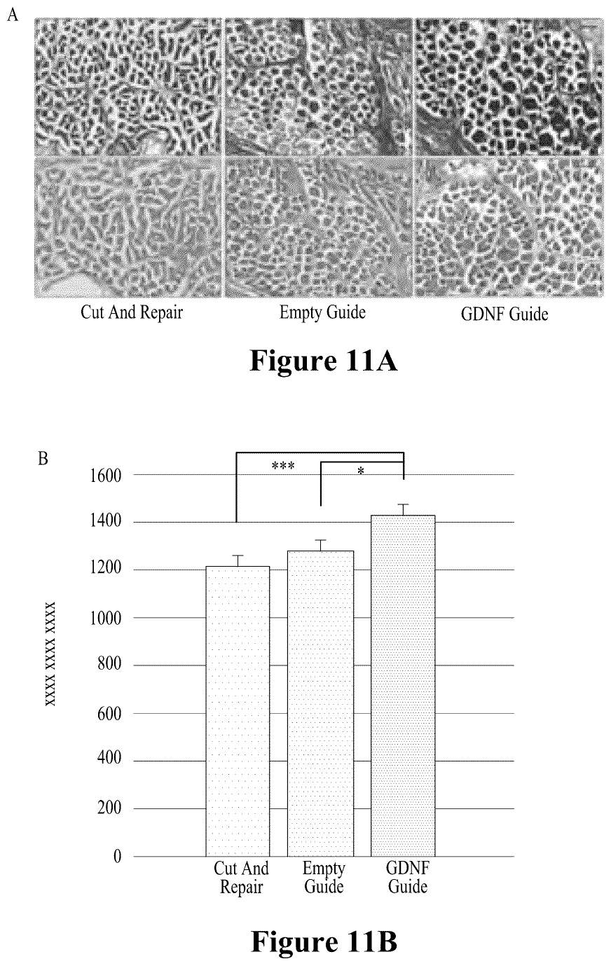

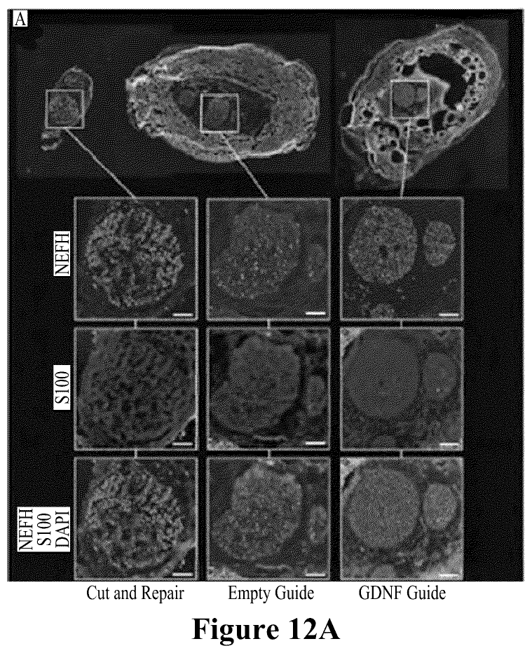

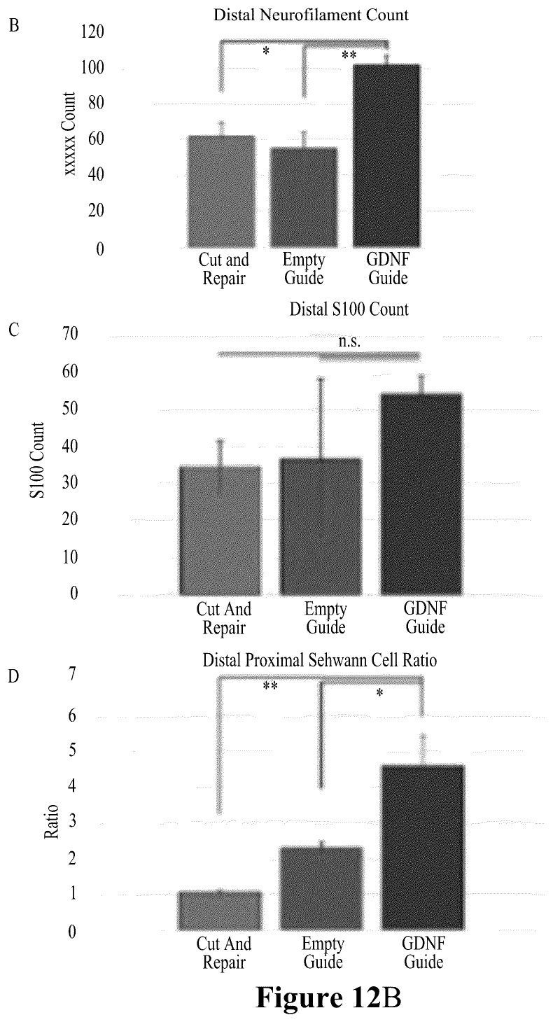

The present example shows GDNF-containing guides demonstrated the highest mean fiber surface area of the target muscle, axonal count of the injured branch, and the number of Schwann cells compared to other guides or treatments. The GDNF-treated group had the highest mean muscle fiber surface area of the levator labii superioris muscles (p<0.05 vs. empty guide, p<0.001 vs. cut and repair only) (FIGS. 11A and 11B). The morphology of the neural tissues was preserved in all experimental conditions. Uninjured nerves from the contralateral side subjectively had similar axonal morphology among experimental conditions while being different from the injured sides. The GDNF-treated group demonstrated the highest axonal count versus others on the region distal to the injury (p<0.01 vs. empty guide, p<0.05 vs. cut and repair only) (FIGS. 12A and 12B). Compared with the proximal segments, the ratio of Schwann cells was significantly higher in the area distal to the injury (p<0.05 vs. empty guide, p<0.01 vs. cut and repair only) in the GDNF-treated group (FIGS. 12C and 12D).

### Discussion

The biodegradable nerve guide containing double-walled GDNF microspheres enhanced recovery after facial nerve transection in rats. The use of a GDNF-containing nerve guide achieved higher muscle action potentials and amplitudes of whisking at earlier time points. The sustained release of exogenous GDNF recruited Schwann cells to the site of ongoing degeneration following injury. At the 12-week timepoint, despite having both end segments of the nerve contained within the GDNF-containing nerve guide, there was an increased number of S100 positive Schwann cells on the region distal to the injury. The presented findings corroborate the current research outcomes when employing localized GDNF delivery for nerve injuries.

Studies in rodent facial nerve injury models have previously examined the potential of GDNF to enhance nerve regeneration and functional recovery²⁴. PLGA nerve conduits containing Schwann cells transfected with GDNF⁴⁹ and synthetic nerve guides containing GDNF²¹ were used in rat models of buccal branch nerve gap with improved outcomes relative to controls. In another study, a nerve guide with GDNF-releasing channels in a 10-mm gap model of the facial nerve was used with immediate and delayed repair²². Despite showing improved recovery with the use of the nerve guide 7 months after the injury, immediate use of the nerve guide had deleterious effects with inhibition of nerve regeneration. This was explained by GDNF “overdose” when the exogenous release from the nerve guide is combined with the intrinsic upregulation of GDNF following injury. The present example demonstrated that harmful effects immediately after the injury were mitigated by controlled release from the microspheres.

There are various drug delivery methods to provide controlled release of growth factors to an injured nerve segment⁵⁰. The microspheres used in the present example have been previously characterized for their release kinetics³⁷⁻⁴⁰. Following the initial burst, GDNF was released with near zero-order kinetics up to approximately 120 days in vitro. Almost 90% of the drug released around day 60. This is a distinct release profile compared with the typical burst release profile seen in single-walled microspheres²⁹. The extended release was achieved by slower degradation of the PLLA shell compared to the PLGA core containing the GDNF. In the present example, the total amount of GDNF encapsulated into the microspheres was also characterized. Implanted conduits contained 222 ng of GDNF per guide. The group with the GDNF guide performed better than the control conditions.

PCL nerve guides without additional cues have been shown to improve recovery for nerve gaps⁵¹, however, the present example shows that such effects were not observed with the use of the empty nerve guide. This is an expected outcome considering that the “gold standard” treatment was already performed in all the experimental conditions (i.e., the tension-free primary repair of the injured nerve). Following tension free primary repair, the addition of a slowly degrading porous polymer construct did not change the outcome significantly and performed as well as the cut and repair treatment. These results demonstrate that the improvement observed in the GDNF-treated group was particularly specific to the drug-embedded microspheres itself, rather than the construct.

The anatomical similarity of the facial nerve branching pattern between humans and rodents and the ability to track the whisking movements in rodents provided a clinically relevant model^{44,52,53}. Movements of the vibrissal muscles are primarily controlled by the innervation of the buccal branch of the facial nerve^{47,54-56}. Both the marginal mandibular and the buccal branches can contribute nearly 100% to the whisker movements, after isolated transection of either branch⁴¹. If both branches are transected, such as the presently disclosed rat model, the amplitude of whisking is expected to be <1% of the initial values, until the regeneration of either of the branches occurs. Therefore, recent studies utilized only one branch to study recovery and resected a segment from the other branch to prevent the second branch from healing^{48,57,58}.

Despite providing a controlled test bed, the above-mentioned approach limited the overall amplitude angle at the long-term follow-up. Previous studies have shown that the functional whisking recovery after injury fails to reach half of the initial values before the injury^{59,60}. The ideal window to study nerve recovery was shown to fall within the first 4 months following injury, where the improvement beyond that point remains unchanged⁶¹. The present example demonstrated that initial recovery in all groups was observed starting from week 3, peaking around week 6, and maintaining a relatively constant amplitude angle beyond week 6. The present example further accounted for the intrinsic differences between rats by normalizing the injured side whisking amplitude by the initial values before the surgery and the values from the contralateral uninjured side. The GDNF-treated group had significantly higher recovery at week 6, compared to the week 1, while previous efforts failed to achieve a significant recovery at the same time point.

### CONCLUSION

The present disclosure demonstrated a biodegradable nerve guide containing a double-walled GDNF microsphere enhanced recovery in a rat facial nerve transection model as assessed by earlier peak whisker movements, higher muscle action potentials, and higher axonal and Schwann cell counts.

### REFERENCES

1. Bradbury ET, Simons W, Sanders R. Psychological and social factors in reconstructive surgery for hemi-facial palsy. J Plast Reconstruct Aesthet Surg. 2006; 59(3):272-278.
2. Coulson SE, O'Dwyer NJ, Adams RD, Croxson GR. Expression of emotion and quality of life after facial nerve paralysis. Otol Neurotol. 2004; 25(6):1014-1019.
3. Hohman MH, Hadlock TA. Etiology, diagnosis, and management of facial palsy: 2000 patients at a facial nerve center. Laryngoscope. 2014; 124(7). doi:10.1002/lary.24542
4. Gordin E, Lee TS, Ducic Y, Arnaoutakis D. Facial nerve trauma: evaluation and considerations in management. Craniomaxillofac Trauma Reconstr. 2015; 8(1):1-13.
5. Lorch M, Teach SJ. Etiology and approach to diagnosis and treatment. Pediatr Emerg Care. 2010; 26(10):10.
6. Spencer CR, Irving RM. Causes and management of facial nerve palsy. Br J Hosp Med. 2016; 77(12):686-691.
7. Owusu JA, Stewart CM, Boahene K. Facial nerve paralysis. Med Clin North Am. 2018; 102(6):1135-1143.
8. Sahovaler A, Yeh D, Yoo J. Primary facial reanimation in head and neck cancer. Oral Oncol. 2017; 74(August):171-180.
9. Razfar A, Lee MK, Massry GG, Azizzadeh B. Facial paralysis reconstruction. Otolaryngol Clin North Am. 2016; 49(2):459-473.
10. Condie D, Tolkachjov SN. Facial nerve injury and repair. Dermatol Surg. 2019; 45(3):340-357.
11. Rosson GD, Redett RJ. Facial palsy: anatomy, etiology, grading, and surgical treatment. J Reconstruct Microsurg. 2008; 24(6):379-389.
12. Lee LN, Lyford-Pike S, Boahene KDO. Traumatic facial nerve injury. Otolaryngol Clin North Am. 2013; 46(5):825-839.
13. Frijters E, Hofer SOP, Mureau MAM. Long-term subjective and objective outcome after primary repair of traumatic facial nerve injuries. Ann Plast Surg. 2008; 61(2):181-187.
14. Navissano M, Malan F, Carnino R, Battiston B. Neurotube for facial nerve repair. Microsurgery. 2005; 25(4):268-271.
15. Inada Y, Hosoi H, Yamashita A, et al. Regeneration of peripheral motor nerve gaps with a polyglycolic acid-collagen tube: technical case report. Neurosurgery. 2007; 61(5):E1105-E1107; discussion E1107.
16. Nakamura Y, Takanari K, Ebisawa K, Kanbe M, Nakamura R, Kamei Y. Repair of temporal branch of the facial nerve with novel polyglycolic acid-collagen tube: a case report of two cases. Nagoya J Med Sci. 2020; 82(1):123-128.
17. Gaudin R, Knipfer C, Henningsen A, Smeets R, Heiland M, Hadlock T. Approaches to peripheral nerve repair: generations of biomaterial conduits yielding to replacing autologous nerve grafts in craniomaxillofacial surgery. BioMed Res Int. 2016; 2016. doi:10.1155/2016/3856262
18. Moskow J, Ferrigno B, Mistry N, et al. Review: bioengineering approach for the repair and regeneration of peripheral nerve. Bioact Mater. 2019; 4(1):107-113.
19. Wang T V., Delaney S, Pepper JP. Current state of stem cell-mediated therapies for facial nerve injury. Curr Opin Otolaryngol Head Neck Surg. 2016; 24(4):285-293.
20. Kishimoto N, Honda Y, Momota Y, Tran SD. Dedifferentiated Fat (DFAT) cells: a cell source for oral and maxillofacial tissue engineering. Oral Dis. 2018; 24(7):1161-1167.
21. Barras FM, Pasche P, Bouche N, Aebischer P, Zurn AD. Glial cell linederived neurotrophic factor released by synthetic guidance channels promotes facial nerve regeneration in the rat. J Neurosci Res. 2002; 70(6):746-755.
22. Barras FM, Kuntzer T, Zurn AD, Pasche P. Local delivery of glial cell linederived neurotrophic factor improves facial nerve regeneration after late repair. Laryngoscope. 2009; 119(5):846-855.
23. Bendella H, Rink S, Grosheva M, Sarikcioglu L, Gordon T, Angelov DN. Putative roles of soluble trophic factors in facial nerve regeneration, target reinnervation, and recovery of vibrissal whisking. Exp Neurol. 2018; 300(August 2017):100-110.
24. Bengur FB, Stoy C, Binko MA, et al. Facial nerve repair: bioengineering approaches in preclinical models. Tissue Eng Part B: Rev. 2022; 28(2):364-378.
25. Dy CJ, Aunins B, Brogan DM. Barriers to epineural scarring: role in treatment of traumatic nerve injury and chronic compressive neuropathy. J Hand Surg. 2018; 43(4):360-367.
26. Fogli B, Corthout N, Kerstens A, et al. Imaging axon regeneration within synthetic nerve conduits. Sci Rep. 2019; 9(1):10095. doi:10.1038/s41598-019-46579-w
27. Nicolas CF, Corvi JJ, Zheng Y, et al. Resorbable nerve wraps: can they be overtightened? J Reconstr Microsurg. 2022; 38(9):694-702.
28. Cintron-Colon A, Almeida-Alves G, VanGyseghem J, Spitsbergen J. GDNF to the rescue: GDNF delivery effects on motor neurons and nerves, and muscle re-innervation after peripheral nerve injuries. Neural Regen Res. 2022; 17(4):748.
29. Tajdaran K, Gordon T, Wood MD, Shoichet MS, Borschel GH. An engineered biocompatible drug delivery system enhances nerve regeneration after delayed repair: biocompatible Drug Delivery System. J Biomed Mater Res. 2016; 104(2):367-376.
30. Iwase T, Jung CG, Bac H, Zhang M, Soliven B. Glial cell line-derived neurotrophic factor-induced signaling in Schwann cells. J Neurochem. 2005; 94(6):1488-1499.
31. Chao T, Pham K, Steward O, Gupta R. Chronic nerve compression injury induces a phenotypic switch of neurons within the dorsal root ganglia. J Comp Neurol. 2008; 506(2): 180-193.
32. Yan Q, Matheson C, Lopez OT. In vivo neurotrophic effects of GDNF on neonatal and adult facial motor neurons. Nature. 1995; 373(6512):341-344.
33. Fine EG, Decosterd I, Papaloi “zos M, Zurn AD, Aebischer P. GDNF and NGF released by synthetic guidance channels support sciatic nerve regeneration across a long gap: GDNF and NGF support nerve regeneration across long gaps. Eur J Neurosci. 2002; 15(4):589-601.
34. Höke A. Schwann cells express motor and sensory phenotypes that regulate axon regeneration. J Neurosci. 2006; 26(38):9646-9655.
35. Höke A, Gordon T, Zochodne DW, Sulaiman OAR. A decline in glial cell line-derived neurotrophic factor expression is associated with impaired regeneration after long-term schwann cell denervation. Exp Neurol. 2002; 173(1):77-85.
36. Streppel M, Azzolin N, Dohm S, et al. Focal application of neutralizing antibodies to soluble neurotrophic factors reduces collateral axonal branching after peripheral nerve lesion: trophic factors and target reinnervation. Eur J Neurosci. 2002; 15(8):1327-1342.
37. Kokai LE, Ghaznavi AM, Marra KG. Incorporation of double-walled microspheres into polymer nerve guides for the sustained delivery of glial cell line-derived neurotrophic factor. Biomaterials. 2010; 31(8):2313-2322.
38. Kokai LE, Bourbeau D, Weber D, McAtee J, Marra KG. Sustained growth factor delivery promotes axonal regeneration in long gap peripheral nerve repair. Tissue Eng Part A. 2011; 17(9-10):1263-1275.
39. Bliley JM, Sivak WN, Minteer DM, et al. Ethylene oxide sterilization preserves bioactivity and attenuates burst release of encapsulated glial cell line derived neurotrophic factor from tissue engineered nerve guides for long gap peripheral nerve repair. ACS Biomater Sci Eng. 2015; 1(7):504-512.
40. Fadia NB, Bliley JM, DiBernardo GA, et al. Long-gap peripheral nerve repair through sustained release of a neurotrophic factor in nonhuman primates. Sci Transl Med. 2020; 12(527):eaav7753.
41. Henstrom D, Hadlock T, Lindsay R, et al. The convergence of facial nerve branches providing whisker pad motor supply in rats: implications for facial reanimation study: rat Facial Nerve Anatomy. Muscle Nerve. 2012; 45(5):692-697.
42. Tomov T, Guntinas-Lichius O, Grosheva M, et al. An example of neural plasticity evoked by putative behavioral demand and early use of vibrissal hairs after facial nerve transection. Exp Neurol. 2002; 178(2):207-218.
43. Abbas OL, Borman H, Uysal CxA, et al. Adipose-derived stem cells enhance axonal regeneration through cross-facial nerve grafting in a rat model of facial paralysis. Plast

Reconstruct Surg. 2016; 138(2):387-396.
44. Guntinas-Lichius O, Irintchev A, Streppel M, et al. Factors limiting motor recovery after facial nerve transection in the rat: combined structural and functional analyses. Eur J Neurosci. 2005; 21(2):391-402.
45. Hadlock TA, Kim SW, Weinberg JS, Knox CJ, Hohman MH, Heaton JT. Quantitative analysis of muscle histologic method in rodent facial nerve injury. JAMA Facial Plast Surg. 2013; 15(2):141-146.
46. Haidarliu S, Simony E, Golomb D, Ahissar E. Muscle architecture in the mystacial pad of the rat. Anat Rec. 2010; 293(7):1192-1206.
47. Dörfl J. The innervation of the mystacial region of the white mouse: a topographical study. J Anat. 1985; 142:173-184.
48. Matsumine H, Numakura K, Climov M, Watanabe Y, Giatsidis G, Orgill DP. Facial-nerve regeneration ability of a hybrid artificial nerve conduit containing uncultured adipose-derived stromal vascular fraction: an experimental study. Microsurgery. 2017; 37(7):808-818.
49. Zhou L, Du HD, Tian H Bin, Li C, Tian J, Jiang JJ. Experimental study on repair of the facial nerve with Schwann cells transfected with GDNF genes and PLGA conduits. Acta Oto-Laryngol. 2008; 128(11):1266-1272.
50. Lee AC, Yu VM, Lowe JB, et al. Controlled release of nerve growth factor enhances sciatic nerve regeneration. Exp Neurol. 2003; 184(1):295-303.
51. Reid AJ, de Luca AC, Faroni A, et al. Long term peripheral nerve regeneration using a novel PCL nerve conduit. Neurosci Lett. 2013; 544:125-130.
52. Angelov DN, Guntinas-Lichius O, Wewetzer K, Neiss WF, Streppel M. Axonal branching and recovery of coordinated muscle activity after transection of the facial nerve in adult rats. Adv Anat Embryol Cell Biol. 2005; 180:1-130.
53. Ozcan G, Shenaq S, Spira M. Vascularized nerve tube: an experimental alternative for vascularized nerve grafts over short gaps. J Reconstruct Microsurg. 1993; 9(6):405-413.
54. Berg RW, Kleinfeld D. Rhythmic whisking by rat: retraction as well as protraction of the vibrissae is under active muscular control. J Neurophysiol. 2003; 89(1):104-117.
55. Bermejo R, Houben D, Zeigler HP. Optoelectronic monitoring of individual whisker movements in rats. J Neurosci Methods. 1998; 83(2):89-96.
56. Heaton JT, Kowaleski JM, Bermejo R, Zeigler HP, Ahlgren DJ, Hadlock TA. A system for studying facial nerve function in rats through simultaneous bilateral monitoring of eyelid and whisker movements. J Neurosci Methods. 2008; 171(2):197-206.
57. Fujimaki H, Matsumine H, Osaki H, et al. Dedifferentiated fat cells in polyglycolic acid-collagen nerve conduits promote rat facial nerve regeneration. Regen Ther. 2019; 11:240-248.
58. Sasaki R, Matsumine H, Watanabe Y, et al. Electrophysiologic and functional evaluations of regenerated facial nerve defects with a tube containing dental pulp cells in rats. Plast Reconstruct Surg. 2014; 134(5):970-978.
59. Kleiss IJ, Knox CJ, Malo JS, Marres HAM, Hadlock TA, Heaton JT. Whisking recovery after automated mechanical stimulation during facial nerve regeneration. JAMA Facial Plast Surg. 2014; 16(2133-139.
60. Heaton JT, Sheu SH, Hohman MH, et al. Rat whisker movement after facial nerve lesion: evidence for autonomic contraction of skeletal muscle. Neuroscience. 2014; 265:9-20.
61. Banks CA, Knox C, Hunter DA, Mackinnon SE, Hohman MH, Hadlock TA. Long-term functional recovery after facial nerve transection and repair in the rat. J Reconstruct Microsurg. 2015; 31(3):210-216.
62. Kasukurthi R, Brenner MJ, Moore AM, et al. Transcardial perfusion versus immersion fixation for assessment of peripheral nerve regeneration. J Neurosci Methods. 2009; 184(2):303-309.
63. Costa HJZR, Ferreira Bento R, Salomone R, et al. Mesenchymal bone marrow stem cells within polyglycolic acid tube observed in vivo after six weeks enhance facial nerve regeneration. Brain Res. 2013; 1510:10-21.
64. Passipieri JA, Dienes J, Frank J, et al. Adipose stem cells enhance nerve regeneration and muscle function in a peroneal nerve ablation model. Tissue Eng Part A. 2021; 27(5-6):297-310.

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

What is claimed is:

1. A system for coating a polymer layer with particles comprising:
a base, wherein the base comprises a flat surface and a gear track;
a removable frame coupled to the plate surface of the base, wherein a layer of particles is formed within the frame; and
a mandrel base, wherein a polymer coated mandrel is attached to the mandrel base;
wherein the mandrel base rotates in the gear track in a direction perpendicular to the flat surface with the polymer coated mandrel rotating on the layer of particles to form a particle coated polymer layer.

2. The system of claim 1, further comprising:
a removable mandrel guide coupled to the base, wherein the mandrel guide forms a channel with the flat surface;
when the mandrel base rotates in the gear track, the polymer coated mandrel rotates in the channel.

3. The system of claim 1, further comprising:
a spreader, wherein the spreader spreads the particles on the flat surface within the frame to form the layer of particles.

4. The system of claim 1, further comprising:
a removable parchment holder coupled to the base, wherein the parchment holder holds wax paper or parchment paper on the flat surface, and
wherein the layer of particles is formed on the wax paper or parchment paper.

5. The system of claim 1, wherein the removable frame is coupled to the flat surface via magnet.

6. The system of claim 1, wherein the gear track is a groove.

7. The system of claim 1, wherein the particles are selected from the group consisting of a microsphere, a nanosphere, and a combination thereof.

8. The system of claim 1, wherein the particles comprise double-walled particles, wherein the double-walled particles comprise the active agent that is released over a pre-determined period of time.

9. The system of claim 8, wherein the double-walled particles comprise a core and shell, wherein the core comprises poly(lactic-co-glycolic acid) (PLGA) and the shell comprises poly(L-lactide) (PLLA).

10. The system of claim 8, wherein the double-walled particles further comprise active agent, and wherein the active agent is Glial Cell Line-Derived Neurotrophic Factor (GDNF).

11. The system of claim 10, wherein a drug dose of the neurotrophic factor in the double-walled particles is from about 3 ng/mg to about 6 ng/mg.

12. The system of claim 1, wherein the mandrel is selected from the group consisting of a biodegradable structure, a biologically derived structure, a bioactive structure, and combinations thereof.

13. The system of claim 1, wherein the mandrel comprises a cylindrical structure of purified collagen or a decellularized scaffold.

14. The system of claim 1, wherein the polymer is polycaprolactone (PCL).

15. The system of claim 1, wherein the system is made from polylactic acid (PLA) or steel.

16. A implantable device for repairing or regenerating a nerve defect in a subject, prepared by the system of claim 1.

17. A method for repairing or regenerating a nerve defect, comprising: placing a cylindrical medical device around the nerve defect.

18. The method of claim 17, wherein the cylindrical medical device comprises:
a plurality layer of polymer.
wherein at least one of the plurality of polymer layers comprises a coating of a plurality of particles.

19. The method of claim 18. wherein the polymer is polycaprolactone (PCL).

20. The method of claim 18. wherein the particles comprise double-walled particles.
